(19) United States
(12) Patent Application Publication
(10) Pub. No.: US 2024/0415820 A1
(43) Pub. Date: Dec. 19, 2024

(54) COMBINATIONS FOR RETAINING FOLLICLES RESERVE

(71) Applicant: SHEBA IMPACT LTD., Ramat Gan (IL)

(72) Inventor: Dror MEIROW, Jerusalem (IL)

(21) Appl. No.: 18/702,029

(22) PCT Filed: Oct. 16, 2022
(86) PCT No.: PCT/IL2022/051093

§ 371 Date: Apr. 17, 2024

Related U.S. Application Data
(60) Provisional application No. 63/256,612, filed on Oct. 17, 2021.

(51) Int. Cl. A61K 31/439; A61K 38/22; A61P 15/08

(52) U.S. Cl. CPC A61K 31/439; A61K 38/22; A61P 15/08

(57) ABSTRACT

The present disclosure provides combinations inhibitors of phosphatidylinositol 3-kinase (PI3K) pathway and agonists of anti-Mullerian hormone receptor type 2 (AMHR2), compositions and kits comprising the same, methods and uses thereof for retaining Primordial Follicle (PMF) reserve and increasing fertility.

### TECHNOLOGICAL FIELD

The present invention relates to combinations, compositions, uses and methods for retaining follicles reserve and/or increasing fertility.

### BACKGROUND ART

References considered to be relevant as background to the presently disclosed subject matter are listed below:

Adhikari D, Liu K. Molecular mechanisms underlying the activation of mammalian primordial follicles. Endocr Rev 2009; 30(5): 438-464.

Anderson R A, Spears N. Cancer treatment and the ovary, Clinical and laboratory analysis of ovarian toxicity. Academic Press. 2015

Bath L E, Wallace W H, Shaw M P, Fitzpatrick C, Anderson R A. Depletion of ovarian reserve in young women after treatment for cancer in childhood: detection by anti-Müllerian hormone, inhibin B and ovarian ultrasound. Hum Reprod. 2003; (11):2368-2374.

Bedenk J, Vrtac̆nik-Bokal E, Virant-Klun I. The role of anti-Millerian hormone (AMH) in ovarian disease and infertility. J Assist Reprod Genet. 2020; 37(1):89-100.

Bellusci G, Mattiello L, Iannizzotto V, Ciccone S, Maiani E, Villani V, Diederich M, Gonfloni S. Kinase-independent inhibition of cyclophosphamide-induced pathways protects the ovarian reserve and prolongs fertility. Cell Death Dis. 2019; 10(10): 726
Bozec A, Etienne-Grimaldi M C, Fischel J L, Sudaka A, Toussan N, Formento P, Milano G. The mTOR-targeting drug temsirolimus enhances the growth-inhibiting effects of the cetuximab-bevacizumab-irradiation combination on head and neck cancer xenografts. Oral Oncol. 2011; 47:340-344.
Carlsson I B, Scott J E, Visser J A, Ritvos O, Themmen A P, Hovatta O. Anti-Mullerian hormone inhibits initiation of growth of human primordial ovarian follicles in vitro. Hum Reprod. 2006; 21(9):2223-7.
Castrillon D H, Miao L, Kollipara R, Homer J W, DePinho R A. Suppression of ovarian follicle activation in mice by the transcription factor Foxo3a. Science 2003; 301: 215-8.
Chang E M, Lim E, Yoon S, Jeong K, Bae S, Lee D R, Yoon T K, Choi Y, Lee W S.

Cisplatin induces overactivation of the dormant primordial follicle through PTEN/AKT/FOXO3a pathway which leads to loss of ovarian reserve in mice. PLoS One 2015; 10(12): e0144245.
Donnez J, Martinez-Madrid B, Jadoul P, Van Langendonckt A, Demylle D, Dolmans M M. Ovarian tissue cryopreservation and transplantation: A review. Hum. Reprod. Update. 2006; 12: 519-535.
Durlinger A L, Gruijters M J, Kramer P, Karels B, Ingraham H A, Nachtigal M W, Uilenbroek J T, Grootegoed J A, Themmen A P. Anti-Müllerian hormone inhibits initiation of primordial follicle growth in the mouse ovary. Endocrinology. 2002; 143(3):1076-84
Durlinger A L, Kramer P, Karels B, De Jong F H, Uilenbroek J T, Grootegoed J A, Themmen A P. Control of primordial follicle recruitment by anti-MUllerian hormone in the mouse ovary, Endocrinology. 1999; 140(12): 5789-96.
Eldani M, Luan Y, Xu P. C, Bargar T, Kim S. Y. Continuous treatment with cisplatin induces the oocyte death of primordial follicles without activation. FASEB J. 2020: 34(10): 13885-13899.
Gigli I, Cushman R A, Wahl C M, Fortune J E. Evidence for a role for anti-Mullerian hormone in the suppression of follicle activation in mouse ovaries and bovine ovarian cortex grafted beneath the chick chorioallantoic membrane. Mol Reprod Dev. 2005 71(4): 480-488.
Goldman K N, Chenette D, Arju R, Duncan F E, Keefe D L, Grifo J A, Schneider R J. mTORC1/2 inhibition preserves ovarian function and fertility during genotoxic chemotherapy. Proc. Natl. Acad. Sci. USA. 2017; 114: 3186-3191.
Gonfloni S, Di Tella L, Caldarola S, Cannata S M, Klinger F G, Di Bartolomeo C, Mattei M, Candi E, De Felici M, Melino G, Cesareni G. Inhibition of the c-Abl-TAp63 pathway protects mouse oocytes from chemotherapy-induced death. Nat Med. 2009; 15(10): 1179-1185.
Gracia C R, Sammel M D, Freeman E, Prewitt M, Carlson C, Ray A, Vance A, Ginsberg J P. Impact of cancer therapies on ovarian reserve. Fertil Steril 2012; 97(1): 134-140.
Hovatta O, Wright C, Krausz T, Hardy K, Winston R M. Human primordial, primary and secondary ovarian follicles in long-term culture: effect of partial isolation. Hum Reprod. 1999; 14(10):2519-2524
Jang H, Lee O H, Lee Y, Yoon H, Chang E M, Park M, Lee J W, Hong K, Kim J O, Kim N K, Ko J J, Lee D R, Yoon T K, Lee W S, Choi Y. Melatonin prevents cisplatin-induced primordial follicle loss via suppression of PTEN/AKT/FOXO3a pathway activation in the mouse ovary. J Pineal Res 2016; 60(3): 336
Jayasinghe Y L, Wallace W H B, Anderson R A. Ovarian function, fertility and reproductive lifespan in cancer patients. Expert Rev Endocrinol Metab. 2018; 13(3):125-136.
John G B, Gallardo T D, Shirley U, Castrillon D H. Foxo3 is a PI3K-dependent molecular switch controlling the initiation of oocyte growth. Dev Biol 2008; 321(1): 197-204.
Kalich-Philosoph L, Roness H, Carmely A, Fishel-Bartal M, Ligumsky H, Paglin S, Wolf I, Kanety H, Sredni B, Meirow D. Cyclophosphamide triggers follicle activation and “burnout”; AS101 prevents follicle loss and preserves fertility. Sci. Transl. Med. 2013; 5: 185ra62
Kano M, Sosulski A E, Zhang L, Saatcioglu H D, Wang D, Nagykery N, Sabatini M E, Gao G, Donahoe P K, Pdpin, D. AMH/MIS as a contraceptive that protects the ovarian reserve during chemotherapy. Proc Natl Acad Sci USA 2017; 114(9): E1688-E1697.
Kawamura K, Cheng Y, Suzuki N, Deguchi M, Sato Y, Takae S, Ho C, Kawamura N, Tamura M, Hashimoto S. Hippo signaling disruption and Akt stimulation of ovarian follicles for infertility treatment. Proc Natl Acad Sci. 2013; 110:17474-17479.
Klinger F G, Rossi V, De Felici M. Multifaceted programmed cell death in the mammalian fetal ovary. Int J Dev Biol. 2015; 59: 51-54
Maidarti M, Anderson R A, Telfer E E. Crosstalk between PTEN/PI3K/Akt signaling and DNA damage in the oocyte: Implications for primordial follicle activation, oocyte quality and ageing. Cells. 2020; 9(1): 200
Manning B D, Cantley L C. AKT/PKB signaling: Navigating downstream. Cell. 2007; 129: 1261-1274.
McLaughlin E A, McIver S C. Awakening the oocyte: controlling primordial follicle development. Reproduction 2009; 137(1):1-11.
Meirow D, Lewis H, Nugent D, Epstein M. Subclinical depletion of primordial follicular reserve in mice treated with cyclophosphamide: clinical importance and proposed accurate investigative tool. Hum Reprod 1999; 14(7): 1903-1907.
Mita M, Mita A, Rowinsky E K. mTOR inhibition for cancer therapy: Past, present and future. Springer. 2016
Nagamatsu G, Shimamoto S, Hamazaki N, Nishimura Y, Hayashi K. Mechanical stress accompanied with nuclear rotation is involved in the dormant state of mouse oocytes. Sci Adv 2019; 5: eaav9960.
Nguyen Q N, Zerafa N, Liew S H, Findlay J K, Hickey M, Hutt K J. Cisplatin- and cyclophosphamide-induced primordial follicle depletion is caused by direct damage to oocytes. Mol. Hum. Reprod. 2019; 25: 433-444.
Partridge A H, Ruddy K J, Gelber S, Schapira L, Abusief M, Meyer M, Ginsburg E. Ovarian reserve in women who remain premenopausal after chemotherapy for early stage breast cancer. Fertil Steril 2010; 94(2): 638-644.
Petrek J A, Naughton M J, Case L D, Paskett E D, Naftalis E Z, Singletary S E, Sukumvanich P. Incidence, time course, and determinants of menstrual bleeding after breast cancer treatment: a prospective study. J Clin Oncol. 2006; 24:1045-1051.
Reddy P, Zheng W, Liu K. Mechanisms maintaining the dormancy and survival of mammalian primordial follicles. Trends Endocrinol. Metab. 2010; 21, 96-103.
Reddy P, Adhikari D, Zheng W, Liang S, Hamalainen T, Tohonen V, Ogawa W, Noda T, Volarevic S, Huhtaniemi I, Liu K. PDK1 signaling in oocytes controls reproductive aging and lifespan by manipulating the survival of primordial follicles. Hum Mol Genet 2009; 18(15): 2813-2824.
Roness H, Spector I, Leichtmann-Bardoogo Y, Savino A M, Dereh-Haim S, Meirow D. Pharmacological administration of recombinant human AMH rescues ovarian reserve and preserves fertility in a mouse model of chemotherapy, without interfering with anti-tumoral effects. J Assist Reprod Genet. 2019; 36(9):1793-1803.
Sato Y, Kawamura K. Rapamycin treatment maintains developmental potential of oocytes in mice and follicle reserve in human cortical fragments grafted into immune-deficient mice. Mol Cell Endocrinol. 2020; 15; 504:110694.
Schmidt K L, Kryger-Baggesen N, Byskov A G, Andersen C Y. Anti-Mullerian hormone initiates growth of human primordial follicles in vitro. Mol Cell Endocrinol. 2005; 234:87-93.
Shai D, Aviel-Ronen S, Spector I, Raanani H, Shapira M, Gat I, Roness H, Meirow D. Ovaries of patients recently treated with alkylating agent chemotherapy indicate the presence of acute follicle activation, elucidating its role among other proposed mechanisms of follicle loss. Fertil Steril. 2021; 115(5):1239-1249.
Sklar C A, Mertens A C, Mitby P, Whitton J, Stovall M, Kasper C, Mulder J, Green D, Nicholson H S, Yasui Y, Robison L L. Premature menopause in survivors of childhood cancer: a report from the childhood cancer survivor study. J Natl Cancer Inst 2006; 98(13): 890-896.
breaks and microvascular compromise. Aging (Albany N Y). 2011; 3:782-793.
Sonigo C, Beau I, Grynberg M, Binart N. AMH prevents primordial ovarian follicle loss and fertility alteration in cyclophosphamide-treated mice. FASEB J. 2018; 33, 1278.
Tanaka Y, Kimura F, Zheng L, Kaku S, Takebayashi A, Kasahara K, Tsuji S, Murakami T. Protective effect of a mechanistic target of rapamycin inhibitor on an in vivo model of cisplatin-induced ovarian gonadotoxicity. Exp. Anim. 2018; 67: 493-500.
Verbeke G, Molenberghs G. Linear mixed models for longitudinal data. Springer-Verlag, New York. 2000.
Wan X, Shen N, Mendoza A, Khanna C, Helman L J. CCI-779 inhibits rhabdomyosarcoma xenograft growth by an antiangiogenic mechanism linked to the targeting of mTOR/Hif-1alpha/VEGF signaling. Neoplasia. 2006; 8(5):394-401.
Yang M Y, Cushman R A, Fortune J E. Anti-Mullerian hormone inhibits activation and growth of bovine ovarian follicles in vitro and is localized to growing follicles. Mol Hum Reprod. 2017; 23(5): 282-291.
Zhou L, Xie Y, Li S, Liang Y, Qiu Q, Lin H, Zhang Q. Rapamycin prevents cyclophosphamide-induced over-activation of primordial follicle pool through PI3K/Akt/mTOR Signaling Pathway in vivo. J Ovarian Res 2017; 10(1): 56.

Acknowledgement of the above references herein is not to be inferred as meaning that these are in any way relevant to the patentability of the presently disclosed subject matter.

### BACKGROUND

Premature Ovarian Insufficiency (POI) is caused by multiple reasons and is considered, for example, to be a major side effect of several types of gonadotoxic chemotherapies and is of significant concern to young cancer survivors. POI is caused by chemotherapy-induced depletion of the primordial follicles (PMF), which constitute the ovarian reserve (Meirow et al., 1999; Jayasinghe et al., 2018). It has been suggested that this loss results either from direct induction of DNA damage and subsequent apoptosis, or indirectly via over-recruitment of the dormant PMFs into the pool of growing follicles, leading to diminished follicular reserve (Kalich-Philosoph et al., 2013, Chang et al., 2015; Jang et al., 2016).

While chemotherapy-induced apoptosis in granulosa cells of growing follicles has been unequivocally demonstrated (Gonfloni et al., 2009; Kalich-Philosoph et al., 2013) there is a debate about the role of apoptosis in PMFs. Some in vivo mouse model studies have shown evidence of apoptosis in the PMF population following Cy (Luan et al., 2019; Bellusci et al., 2019; Nguyen et al., 2019), other studies failed to show evidence of apoptosis (Kalich-Philosoph et al., 2013; Goldman et al., 2017; Zhou et al., 2017).

Aside from inducing apoptosis, it has also been proposed that chemotherapy dysregulates the factors which act to maintain dormancy of the PMFs, triggering over activation and growth, followed by death of the PMF population. PMF activation is regulated by growth factors, cytokines and transcription factors, and is controlled by interaction between several signaling pathways (Adhikari and Liu 2009; McLaughlin and McIver 2009).

A major pathway controlling dormancy, is the phosphatidylinositol 3-kinase (PI3K) pathway, in which key upstream molecules PI3K and protein kinase B (AKT) control the phosphorylation of principal modulators Forehead Box 03 (FOXO3a) and Mammalian target of rapamycin (mTOR, Castrillon et al., 2003; John et al., 2008; Adhikari and Liu 2009; Reddy et al., 2009).

Follicle activation is also regulated by paracrine factors such as anti-Müllerian hormone (AMH, Durlinger et al., 1999; Adhikari and Liu 2009; Kawamura et al., 2013) as well as physical properties of the microenvironment such as increased pressure (Nagamatsu et al., 2019) and PMF clustering (Hovatta et al., 1999).

It has been shown in mice that ovotoxic agents Cy and cisplatin increased phosphorylation of Akt, mTOR and FOXO3a, (Kalich-Philosoph et al., 2013; Chang et al., 2015; Jang et al., 2016) triggering excessive follicle activation, and loss of PMFs, resulting in decreased fertility (Kalich-Philosoph et al., 2013; Goldman et al., 2017).

It was also shown that administration of mTOR inhibitors (rapamycin or everolimus) has prevented Cy induced upregulation of the PI3K pathway, partially attenuated PMFs loss and improved fertility outcomes (Goldman et al., 2017; Tanaka et al., 2018; Sato and Kawamura 2020).

Administration of the suppressive agent AMH reduced follicle activation and ovarian reserve depletion and improved fertility in mouse models of Cy (Kano et al., 2017; Roness et al., 2019; Sonigo et al., 2018).

### GENERAL DESCRIPTION

The present disclosure provides in accordance with some aspects, a combination therapy comprising at least two modulators, wherein at least one of the at least two modulators inhibits the phosphatidylinositol 3-kinase (PI3K) pathway and at least one other of the at least two modulators is an agonist of anti-Mullerian hormone receptor type 2 (AMHR2).

The present disclosure provides in accordance with some further aspects, a combination therapy comprising at least one inhibitor of the phosphatidylinositol 3-kinase (PI3K) pathway and at least one agonist of anti-Mullerian hormone receptor type 2 (AMHR2).

The present disclosure provides in accordance with other aspects, a combination therapy for use in retaining primordial follicles (PMF) reserve in a subject.

The present disclosure provides in accordance with other aspects, a combination therapy for use in increasing fertility in a subject.

The present disclosure provides in accordance with yet other aspects, a method for retaining PMF reserve in a subject, the method comprising administering to the subject a therapeutically effective amount of a combination comprising at least two modulators, wherein at least one of the at least two modulators is an mTOR inhibitor and at least one other of the at least two modulators is an agonist of AMHR2.

The present disclosure provides in accordance with yet further aspects, a method for increasing fertility in a subject, the method comprising administering to the subject in need thereof a therapeutically effective amount of a combination comprising at least two modulators, wherein at least one of the at least two modulators i is an mTOR inhibitor and at least one other of the at least two modulators is an agonist of AMHR2.

The present disclosure provides in accordance with yet further aspects, a pharmaceutical composition comprising a combination for use in retaining PMF and/or increasing fertility in a subject in need thereof, wherein the combination comprising at least one inhibitor of the phosphatidylinositol 3-kinase (PI3K) pathway and at least one agonist of anti-Mullerian hormone receptor type 2 (AMHR2).

The present disclosure provides in accordance with yet further aspects, a kit comprising a combination and optionally instructions for using the effective amounts in the combination therapy in a method of retaining PMF and/or increasing fertility in a subject, wherein the combination comprising at least one inhibitor of the phosphatidylinositol 3-kinase (PI3K) pathway and at least one agonist of anti-Mullerian hormone receptor type 2 (AMHR2).

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is an exemplary representation of an in vivo experimental outline dosing regimen, Female 8 week old BALB/c mice were randomly allocated to one of 4 treatment groups (n=8 mice per treatment group): (1) mice were given a single intraperitoneal injection of 200 μl of PBS (2) mice received Temsirolimus (Tem) alone (5 mg/Kg, 5 doses in 7 days before Cy treatment) (3) mice received a single intraperitoneal injection of Cy (150 mg/kg), (4) mice received Tem (5 mg/Kg, 5 doses in 7 days before Cy treatment) followed by a single intraperitoneal injection of Cy (150 mg/kg), in the second unit of in vivo experiments (n=8 mice per treatment group), mice received recombinant human AMH (rAMH) as well (5 μg/mouse, 5 doses in 24 hours—every 6 hours following the administration of Cy), in addition to Tem, Cy, both Tem and Cy or alone; four of the mice were sacrificed 24 hours after Cy treatment and their ovaries removed and either snap frozen in liquid nitrogen for protein analysis or immersed in 4% PFA for cytohistological analysis; the remaining mice were sacrificed 3 or 21 days after Cy treatment, and their ovaries removed, immersed in 4% PFA and processed for follicle counting (H&E staining).

FIGS. 2A and 2B are bar graphs showing the effect of Tem and rAMH respectively, in ex vivo PMFs counting on ovaries from 12 days mice that were placed at an ex-vivo system for 7 days after treatment with PM (20 ng/ml) or the equivalent volume of media with or without Tem or rAMH, ovaries were stained with H&E for PMF count, CON refers to control untreated ovaries * p<0.05 ** p<0.01 *** p<0.001.

FIGS. 3A-3C are bar graphs showing the effect of Tem in in vivo counting; ovaries from 8-week-old mice that were removed 3 or 21 days after a single dose of Cy (150 mg/kg) or the equivalent volume of PBS with or without co-treatment with Tem and were stained with H&E for follicle count; FIG. 3A shows PMFs counting 21 days after Cy treatment: 5-10 ovaries were counted for follicles per group; FIG. 3B growing (primary+follicles) and dormant (primordial) follicles counting 3 days after Cy treatment: 5-7 ovaries were counted for follicles per group, FIG. 3C comparison of the growing/dormant follicles ratio between ovaries removed 3 and 21 days following Cy exposure, * p<0.05 ** p<0.01 *** p<0.001.

FIG. 4 is a bar graph showing the effect of Tem+rAMH in vivo in ovaries from 8-week-old mice that were removed 21 days after a single dose of Cy (150 mg/kg) or the equivalent volume of PBS with or without co-treatment with Tern and/or AMH and were stained with H&E for follicle count: Primordial follicles counting 21 days after Cy treatment. 14-16 ovaries were counted for follicles per group, ***p<0.001

FIGS. 5A-5K inhibition of primary follicle proliferation by Tem, rAMH and their combination, FIGS. 5A-5C are representative images of primary follicles group types of primary follicles, FIG. 5A no proliferation—no staining in any granulosa cells positive for Ki-67; FIG. 5B mild proliferation, 1-2 granulosa cells stained positive for Ki-67 (shown by arrows); FIG. 5C extensive proliferation, more than 3 granulosa cell stained positive for Ki-67 (shown by arrows), FIGS. 5D-5K are images showing inhibition of primary follicle proliferation by Tem, rAMH and their combination after a single dose of Cy (150 mg/kg) or the equivalent volume of PBS, FIGS. 5D and 5E ovaries administered with PBS without and with administration of Cy, respectively; FIGS. 5F and 5G ovaries administered with Tem without and with administration of Cy, respectively; FIGS. 5H and 5I ovaries administered with rAMH without and with administration of Cy, respectively; FIGS. 5J and 5K ovaries administered with Tem and rAMH without and with administration of Cy, respectively; an analysis of ovaries from 8-week-old mice that were removed 24 hours after treatment and sample sections were used for immunohistochemistry and stained for proliferation marker Ki-67.

FIGS. 6A-6D show that combined treatment of Tem and rAMH reduced Cy induced PI3K pathway activation: protein analysis of ovaries from 8-week-old mice that were removed 24 hours after a single dose of Cy (150 mg/kg) or the equivalent volume of PBS with or without co-treatment with Tem and/or AMH, FIG. 6A is a representative exemplary western blot of prpS6 and rpS6, FIG. 6B show the respective intensity of prpS6/rpsS6 ratio from 5 ovaries; FIG. 6C is a representative exemplary western blot of pmTOR and vinculin, FIG. 6D show the respective intensity from the western blot of pmTOR/mTOR ratio from 3 ovaries, respectively* p<0.05 ** p<0.01.

FIGS. 7A and 7B are bar graphs showing the effect of Tem (T) and rAMH (A) in decreasing Cy (C) induced DNA damage and direct oocyte death, Sample sections from ovaries removed 12 h (FIG. 7A) or 24 h (FIG. 7B) after Cy treatment, were stained for DNA damage marker γH2AX antibody (FIG. 7A), and apoptotic marker cPARP antibody (FIG. 7B); Primordial follicle oocytes from one or two sections from 6-8 ovaries per group were evaluated (at least 50 follicles from each group), follicles were classified as positive/negative.

### DETAILED DESCRIPTION OF EMBODIMENTS

Premature ovarian insufficiency (POI) and loss of primordial follicles (PMF) reserve are often related with reduced fertility and infertility. The existing method that are used to retain fertility include invasive procedures such as embryo, oocyte, and ovarian tissue cryopreservation. Such methods are often limited by patient age and status.

The present disclosure provides unique non-invasive means for retaining fertility in a subject by inhibiting (reducing) loss of ovarian follicle reserve and specifically loss of PMF and increasing fertility.

Specifically, the present disclosure is based on the surprising findings that modulating different pathways in ovarian follicle, including pathways that are intrinsic to the ovarian follicle and pathways that are extrinsic to the ovarian follicle have complementary (synergistic) effects in retaining follicles reserve and specifically PMF reserve in a mammalian subject.

An important feature of the presently combination therapy as shown in the Examples below demonstrates that the combination therapy is more effective as compared to each one of the combination's components alone.

Surprisingly, while administration of each one of the combination's components separately partially retained PMF reserve by inhibiting chemotherapy (Cy or PM) effect, the combination of the present invention, completely blocked chemotherapy effect. As shown in the Examples below, in mice that were treated with chemotherapy and received the combination therapy, the number of PMF was almost identical to the number of PMF in mice which were not treated with chemotherapy. This demonstrates the unique effect of the combination therapy to retain PMF reserve despite the effect induced by chemotherapy.

As shown in the Examples below, combinations comprising Temsirolimus (Tem), an exemplary modulator of an intrinsic pathway and anti-Müllerian hormone and specifically a C-terminus region of hAMH, an exemplary modulator of an extrinsic pathway, were successful in retaining PMF reserve by blocking premature (primordial) follicles depletion in response to chemotherapy treatment. As noted herein, the effect of the combination was significantly increased as comparted to each one the combination's components separately.

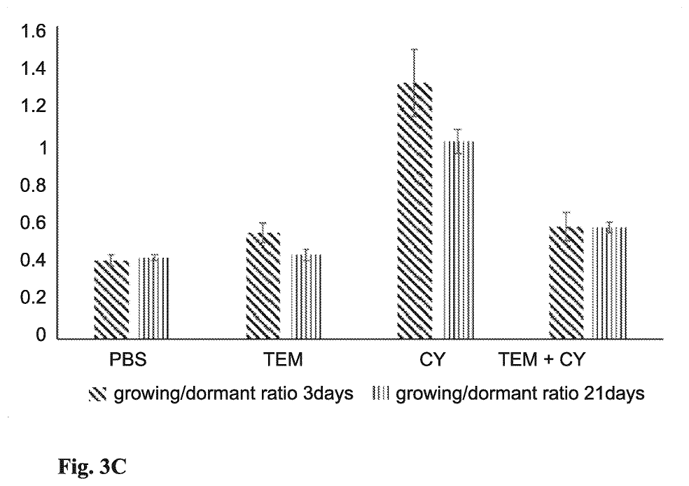

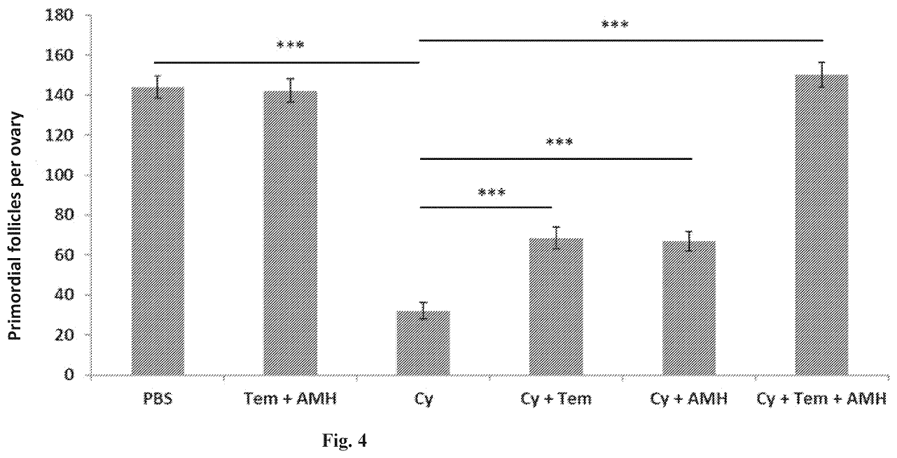

As shown in FIG. 4 below, treatment with chemotherapy alone caused significant loss of PMF reserve. While this loss was partially attenuated with treatment with either Tem or hAMH alone (C-terminal domain of hAMH), the combination therapy (Tem and AMH) provided complete protection of the PMF reserve. As can be seen from the results of FIG. 4, the number of PMFs in mice treated with the combination was almost identical to the number of PMFs in control mice. This suggested that the combination therapy is successful in retaining PMFs reserve.

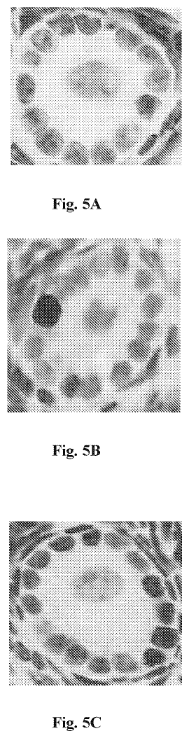

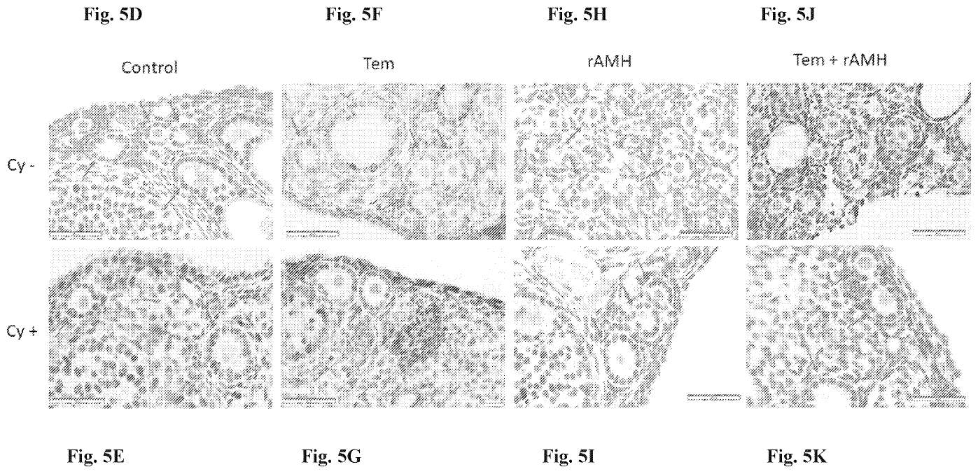

Further, as shown in FIG. 5 below, combinations comprising Tem and hAMH (C-terminal domain of hAMH), inhibited primary follicle proliferation.

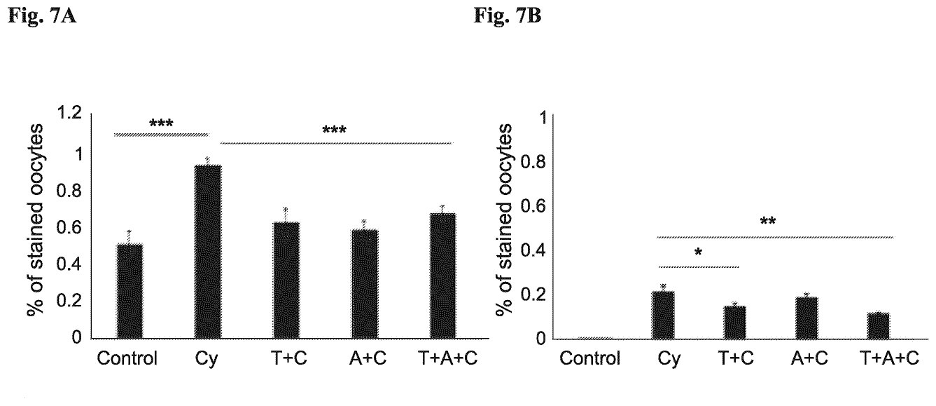

In addition, as shown in FIG. 7 below, staining with γH2AX demonstrated significant DNA-damage in PMF oocytes in mice treated with chemotherapy, and this damage was significantly reduced by treatment with the combination therapy of the invention. The DNA-damage can be attributed to some extend to apoptosis as shown in staining with cPARP. This suggests that the PMF depletion in response to chemotherapy treatment is mainly due to PMF activation and apoptosis.

Without being bound by theory, it was suggested that the combination therapy that comprises a modulator of an intrinsic pathway with a modulator of an extrinsic pathway, including, inter alia, Tem and AMH, respectively, may provide a complementary effect (such as a synergistic effect) in maintaining PMF reserve, in subjects in need to retain PMF reserve, including, inter alia, in subjects treated with chemotherapy

Based on the results, it was envisaged that the combination of the invention that comprises a modulator of an intrinsic pathway with a modulator of an extrinsic pathway, including, inter alia, Tem and AMH, may be applicable in retaining PMF reserve in a subject.

Hence, the invention provides a combination comprising at least two modulators, wherein at least one of the at least two modulators modulate an intrinsic oocyte pathway and at least one of the at least two modulators modulates an external oocyte pathway.

In the following text, when referring to the combinations it is to be understood as also referring to the compositions, methods, uses and kits disclosed herein. Thus, whenever providing a feature with reference to the combinations, it is to be understood as defining the same feature with respect to the compositions, methods, uses and kits, mutatis mutandis.

The term modulator as used herein may be any compound or agent that modulates an intrinsic (internal) oocyte pathway and an extrinsic (external) oocyte pathway. Further, the modular may act directly (e.g., by binding) or indirectly on a receptor or protein, and leads to modulation of its activation, for example, increasing or enhancing its activation, or alternatively inhibiting or decreasing its activation.

In some embodiments, the modulator used by the combinations, compositions, kits, methods and uses of the invention may be an activating modulator that may act as an agonist, and in some embodiments, as a direct antagonist, or as an allosteric agonist, or alternatively, may indirectly activate a step in a pathway.

In some embodiments, the modulator used by the combinations, compositions, kits, methods and uses of the invention may be an inhibiting modulator that may act as an antagonist, and in some embodiments, as a direct antagonist, or as an allosteric inhibitor or alternatively, may indirectly inhibit a step in a pathway.

As further detailed below, each one of the modulators of the invention is or may comprise a synthetic or natural modulator, for example, a nucleic acid modulator, a protein modulator, peptidomimetic modulator, a small molecule modulator, or any combination thereof.

The term an intrinsic oocyte pathway as used herein refers to an intracellular signaling pathway in oocyte or granulosa cells that controls primordial follicles (PMF), which is intrinsic to the oocyte and granulosa cells.

In some embodiments, the intrinsic oocyte pathway is phosphatidylinositol 3-kinase (PI3K) pathway

Hence, the at least one modulator in the combination therapy of the invention is capable of modulating an intrinsic oocyte pathway, including, inter alia, the PI3K pathway.

The phosphatidylinositol 3-kinase (PI3K) pathway include a signaling pathway in which a PI3K activation phosphorylates and activates AKT also known as Protein kinase B (PKB), localizing it in the plasma membrane.

In some embodiments, the combination comprises a modulator of the PI3K pathway.

In accordance with some aspects which can be implemented as embodiments of the invention, it is provided a combination comprising a modulator of the PI3K pathway and at least one modulator of an extrinsic oocyte pathway.

The results shown below (FIG. 5) suggested that the combinations of the invention protected the follicle reservoir by inhibiting upregulation of PIK3 pathway.

Hence, in accordance with some aspects which can be implemented as embodiments of the invention, it is provided a combination comprising an inhibitor of the PI3K pathway and at least one modulator of an extrinsic oocyte pathway.

As appreciated, inhibition of the PI3K pathway by an inhibitor of the PI3K pathway, can be achieved by either activating different factors in the PI3K pathway which suppress PMF or alternatively by inhibiting different factors in the PI3K pathway which activate PMF.

In some embodiments, the modulator is an activator of at least one factor in the PI3K pathway, wherein the at least one factor is capable of suppressing PMF reserve.

In such embodiments, the activator of the PI3K pathway enhances the activity of at least one of phosphatase and tensin homolog (PTEN), FOXO 3a (FOXO3a), protein p27 (p27), Tuberous sclerosis proteins 1/2 (TSC1/TSc2) or any combination thereof.

In some embodiments, the modulator is an inhibitor of at least one factor in the PI3K pathway, wherein the at least one factor is capable of activating PMF reserve.

In such embodiments, the inhibitor of the PI3K pathway inhibits at least one of PI3K, Pyruvate Dehydrogenase Kinase 1 (PDK1), Akt, mammalian target of rapamycin (mTOR), ribosomal protein S6 (rpS6) or any combination thereof.

In some embodiments, the modulator of an intrinsic oocyte pathway acts as an antagonist of the PI3K pathway. In some embodiments, the modulator of an intrinsic oocyte pathway inhibits the PI3K pathway.

In some embodiments, the inhibitor of the PI3K pathway is an inhibitor of mTOR.

The term “antagonist” or “inhibitor” are used interchangeably relates to a compound, agent or a drug that binds to a protein or a receptor and partially or totally blocks stimulation, decreases, prevents, delays activation, inactivates, desensitizes, down regulates the activity or dampens a biological response. In the context of the present disclosure, the antagonist or inhibitor may partially or completely block the activity of mTOR.

The PI3K pathway and specially mTOR related pathway have broad activity, with important cell cycle regulatory roles including direct effects on proliferation, apoptosis, and stem cell differentiation. Interestingly, it was found by the inventors that combinations that comprise modulator of the PI3K pathway and specially mTOR inhibitor were successful in retaining follicle reserve and blocking follicle activation.

In accordance with some aspects which can be implemented as embodiments of the invention, it is provided a combination comprising at least one inhibitor of mTOR and at least one modulator of an extrinsic oocyte pathway.

As appreciated, mTOR also referred to as the mechanistic target of rapamycin, and sometimes called FK506-binding protein 12-rapamycin-associated protein 1 (FRAP1), is a member of phosphatidylinositol 3-kinase-related kinase family of protein kinases and encoded in humans by the MTOR gene.

mTOR refers to a catalytic subunit of two structurally distinct complexes: mTOR Complex 1 (mTORC1) and mTOR Complex 2 (mTORC2).

As appreciated, mTORC1 is composed of mTOR, regulatory-associated protein of mTOR (Raptor), mammalian lethal with SEC13 protein 8 (mLST8) and the non-core components PRAS40 and DEPTOR. The activity of mTORC1 is regulated by various ligands, rapamycin, insulin, growth factors, phosphatidic acid, amino acids and derivatives (e.g., 1-leucine and β-hydroxy β-methylbutyric acid).

As appreciated, mTORC2 is composed of mTOR, rapamycin-insensitive companion of mTOR (RICTOR), MLST8, and mammalian stress-activated protein kinase interacting protein 1 (mSIN1).

The present invention is not limited to a specific inhibitor (antagonist) of mTOR and is applicable to different inhibitors all resulting in partial or complete activity of mTOR.

The at least one mTOR inhibitor may identified and/or designed by methods known in the art, including, inter alia, computational methods. The activity of mTOR inhibitor may be characterized by any method known in the art.

In some embodiments, the mTOR inhibitor inhibits mTOR Complex 1 (mTORC1).

In some embodiments, the mTOR inhibitor inhibits mTOR Complex 2 (mTORC2).

In some embodiments, the mTOR inhibitor is a mTORC1/mTORC2 dual inhibitor. In other words, the mTOR inhibitor inhibits both mTORC1 and mTORC2.

In some embodiments, the mTOR inhibitor is or comprise a pipecolate region.

In some embodiments, the mTOR inhibitor is capable of binding to peptidyl-prolyl cis-trans isomerase (FKBP1A).

In some embodiments, the mTOR inhibitor is capable of binding to mTOR kinase.

In some embodiments, the mTOR inhibitor is capable of binding to ATP-binding site in mTOR kinase.

In some embodiments, the mTOR inhibitor is an ATP-competitive mTOR kinase inhibitor.

In some embodiments, the mTOR inhibitor is capable of binding to peptidyl-prolyl cis-trans isomerase (FKBP1A) and to mTOR kinase.

In some embodiments, the mTOR inhibitor is capable of binding to peptidyl-prolyl cis-trans isomerase (FKBP1A) and to ATP-binding site in mTOR kinase.

In some embodiments, the mTOR inhibitor is a mTOR/PI3K dual inhibitor.

In some embodiments, the mTOR inhibitor is a mTOR/PI3K dual inhibitors, a mTORC1/mTORC2 dual inhibitor or combination thereof.

In some embodiments, the mTOR inhibitor competed with and displaced phosphatidic acid from the FRB domain in mTOR.

In some embodiments, the inhibitor of mTOR is at least one of a small molecule, an aptamer, an antisense RNA, a single-stranded RNA (ssRNA), a double-stranded RNA (dsRNA), a polypeptide, an antibody or fragment thereof or any combination thereof.

In some embodiments, the inhibitor of mTOR is a small molecule.

It should be noted that in accordance with the present disclosure, when referring to a small molecule it may encompasses one or more of the following as well as any combinations thereof: a solvate, a hydrate, a stereoisomer, a pharmaceutically acceptable prodrug, a pharmaceutically active metabolite, a pharmaceutically acceptable salt, a crystalline form, an amorphous form, a physiologically functional derivative of the small molecule.

It should be noted that a solvate, a hydrate, a stereoisomer, a pharmaceutically acceptable prodrug, a pharmaceutically active metabolite, a pharmaceutically acceptable salt, a crystalline form, an amorphous form, a physiologically functional derivative in the context of the present disclosure are considered to have similar biological or physiological activity as the small molecule to which they relate or any small molecule related thereof. As noted herein, the activity of the small molecule in the combinations of the invention are for use and methods for retaining follicle reserve, specifically PMF reserve and/or increase fertility (i.e. reduce infertility).

In some embodiments, the at least one mTOR inhibitor is or comprises at least one rapalog inhibitor.

The term rapalog as used herein refers to rapamycin and synthetic derivatives/analogues of rapamycin. Rapalog inhibitors are at times considered as first generation of mTOR inhibitors.

In some embodiments, the at least one mTOR inhibitor is or comprises at least one non-rapalog inhibitor. Non-rapalog inhibitors are at times considered as non-first generation of mTOR inhibitors.

In some embodiments, the at least one mTOR inhibitor is at least one rapalog inhibitor, at least one non-rapalog inhibitor or combinations thereof.

In accordance with some aspects which can be implemented as embodiments of the invention, it is provided a combination comprising an inhibitor of mTOR and at least one modulator of an extrinsic oocyte pathway, wherein the at least one mTOR inhibitor is or comprises at least one of (i) at least one rapalog inhibitor, (ii) at least one non-rapalog inhibitor or (iii) any combinations thereof.

In accordance with some aspects which can be implemented as embodiments of the invention, it is provided a combination comprising an inhibitor of mTOR and at least one modulator of an extrinsic oocyte pathway, wherein the at least one mTOR inhibitor is or comprises at least one rapalog inhibitor.

In accordance with some aspects which can be implemented as embodiments of the invention, it is provided a combination comprising an inhibitor of mTOR and at least one modulator of an extrinsic oocyte pathway, wherein the at least one mTOR inhibitor is or comprises at least one non-rapalog inhibitor.

In some embodiments, the at least one mTOR inhibitor applicable for the invention is rapamycin or at least one of solvate, a hydrate, a stereoisomer, a pharmaceutically acceptable prodrug, a pharmaceutically active metabolite, a pharmaceutically acceptable salt, a crystalline form, an amorphous form, a physiologically functional derivative of rapamycin or any combinations thereof.

Rapamycin is a small molecule produced by the bacterium Streptomyces hygroscopicus and was isolated from samples of Streptomyces hygroscopicus.

Rapamycin is a molecule having a CAS number 53123-88-9 and is represented by the following chemical structure represented by Formula (I):

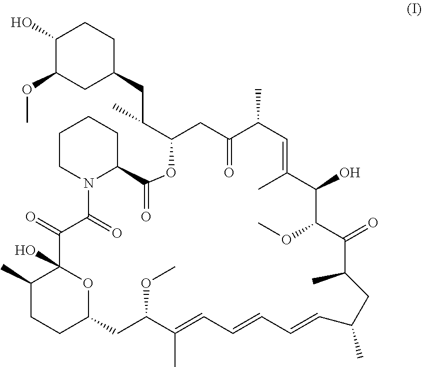 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

In some embodiments, rapamycin derivatives include temsirolimus, everolimus, or ridaforolimus.

In some embodiments, the at least one mTOR inhibitor applicable for the invention is temsirolimus or at least one of solvate, a hydrate, a stereoisomer, a pharmaceutically acceptable prodrug, a pharmaceutically active metabolite, a pharmaceutically acceptable salt, a crystalline form, an amorphous form, a physiologically functional derivative of temsirolimus or any combinations thereof.

Temsirolimus (sold under the brand name Torisel) is a derivative and a prodrug of rapamycin. Specifically, temsirolimus is a dihydroxymethyl propionic acid ester of rapamycin. It is denoted herein at times as Tem.

Temsirolimus is a molecule having a CAS number 162635-04-3 and is represented by the following chemical structure represented by Formula (II):

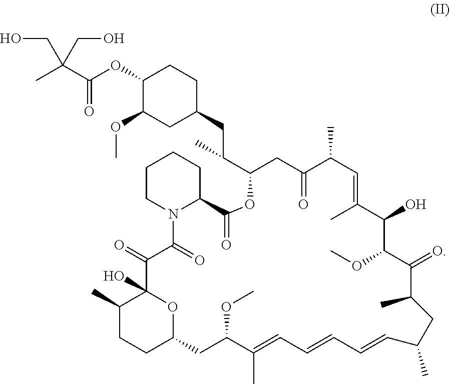 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)C(C)(CO)CO)[C@H](OC)C2)CC(=O)[C@H](C)/C=C(\C)[C@@H](O)[C@@H](OC)C(=O)[C@H](C)C[C@H](C)/C=C/C=C/C=C/1C]

Temsirolimus is at times converted to sirolimus (rapamycin) for example under in vivo conditions.

In some embodiments, the at least one mTOR inhibitor applicable for the invention is everolimus or at least one of solvate, a hydrate, a stereoisomer, a pharmaceutically acceptable prodrug, a pharmaceutically active metabolite, a pharmaceutically acceptable salt, a crystalline form, an amorphous form, a physiologically functional derivative of everolimus or any combinations thereof.

Everolimus (sold under the brand name Afinitor) is a derivative of rapamycin. Specifically, it is the 40-O-(2-hydroxyethyl) derivative as it has O-2 hydroxyethyl chain substitution.

Everolimus is a molecule having a CAS number 159351-69-6. Everolimus is represented by the following chemical structure represented by Formula (III):

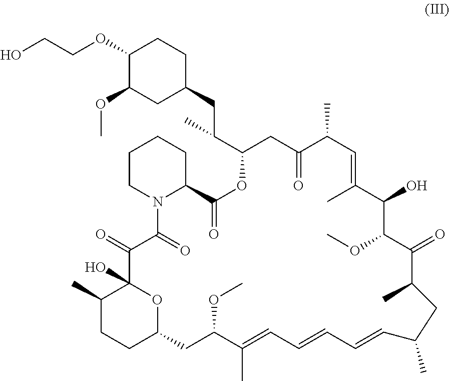 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C]

In some embodiments, the at least one mTOR inhibitor applicable for the invention is ridaforolimus or at least one of solvate, a hydrate, a stereoisomer, a pharmaceutically acceptable prodrug, a pharmaceutically active metabolite, a pharmaceutically acceptable salt, a crystalline form, an amorphous form, a physiologically functional derivative of ridaforolimus or any combinations thereof.

Ridaforolimus (also known as AP23573 and MK-8669; formerly known as deforolimus) is a derivative of rapamycin. Specifically, ridaforolimus has a phosphine oxide substitution at position C-43 in the lactone ring of rapamycin.

Ridaforolimus have a CAS number 572924-54-0. Ridaforolimus is represented by the following chemical structure represented by Formula (IV):

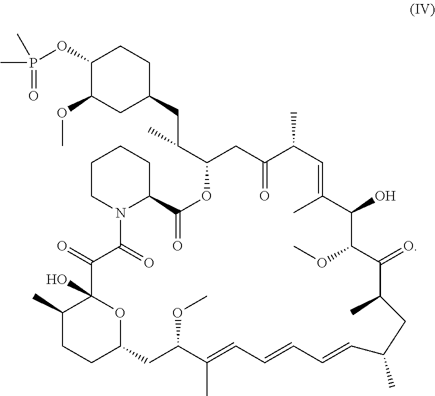 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OP(C)(C)=O)[C@H](OC)C2)CC(=O)[C@H](C)/C=C(\C)[C@@H](O)[C@@H](OC)C(=O)[C@H](C)C[C@H](C)/C=C/C=C/C=C/1C]

In some embodiments, the at least one mTOR inhibitor applicable for the invention is umirolimus or at least one of solvate, a hydrate, a stereoisomer, a pharmaceutically acceptable prodrug, a pharmaceutically active metabolite, a pharmaceutically acceptable salt, a crystalline form, an amorphous form, a physiologically functional derivative of umirolimus or any combinations thereof.

Umirolimus (INN/USAN, also called Biolimus) has a CAS number 851536-75-9. Umirolimus is represented by the following chemical structure represented by Formula (V):

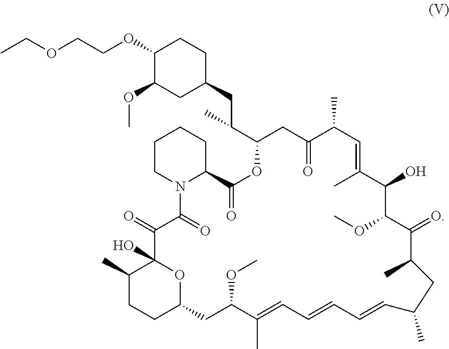 [SMILES: CCOCCO[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC]

In some embodiments, the at least one mTOR inhibitor applicable for the invention is zotarolimus or at least one of solvate, a hydrate, a stereoisomer, a pharmaceutically acceptable prodrug, a pharmaceutically active metabolite, a pharmaceutically acceptable salt, a crystalline form, an amorphous form, a physiologically functional derivative of zotarolimus or any combinations thereof.

Zotarolimus (INN codenamed ABT-578) has a CAS number 221877-54-9. Umirolimus is represented by the following chemical structure represented by Formula (VI):

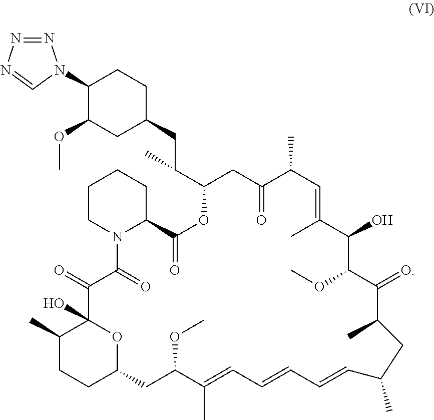 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@H](n3cnnn3)[C@H](OC)C2)CC(=O)[C@H](C)/C=C(\C)[C@@H](O)[C@@H](OC)C(=O)[C@H](C)C[C@H](C)/C=C/C=C/C=C/1C]

In some embodiments, the inhibitor of mTOR is at least one of temsirolimus, rapamycin, everolimus, ridaforolimus or a combination thereof.

As noted herein, reference made to any one of a small molecule being rapamycin, temsirolimus, everolimus, ridaforolimus may refer to at least one of a solvate, a hydrate, a stereoisomer, a pharmaceutically acceptable prodrug, a pharmaceutically active metabolite, a pharmaceutically acceptable salt, a crystalline form, an amorphous form, a physiologically functional derivative of each one of rapamycin, temsirolimus, everolimus or ridaforolimus.

In some embodiments, the mTOR inhibitor is at least one of rapamycin, temsirolimus or a combination thereof.

In some embodiments, the mTOR inhibitor is at least one of rapamycin, everolimus or a combination thereof.

In some embodiments, the mTOR inhibitor is at least one of rapamycin, temsirolimus, everolimus or any combination thereof.

In some embodiments, the mTOR inhibitor is at least one of everolimus ridaforolimus or a combination thereof.

As noted above, the at least one mTOR inhibitor is or comprises in accordance with some embodiments, at least one non-rapalog inhibitor.

In some embodiments, the at least one mTOR inhibitor is a mTOR/PI3K dual inhibitor.

In some embodiments, the at least one mTOR inhibitor is a mTORC1/mTORC2 dual inhibitor.

In some embodiments, the mTOR inhibitor include at least one of Torin-1. Torin-2, vistusertib or any combinations thereof.

In some embodiments, the at least one mTOR inhibitor applicable for the invention is Torin-1 or at least one of solvate, a hydrate, a stereoisomer, a pharmaceutically acceptable prodrug, a pharmaceutically active metabolite, a pharmaceutically acceptable salt, a crystalline form, an amorphous form, a physiologically functional derivative of Torin-1 or any combinations thereof.

Torin-1 has a CAS number 1222998-36-8 and is represented by the following chemical structure represented by Formula (VII):

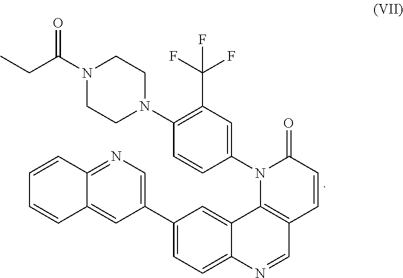 [SMILES: CCC(=O)N1CCN(c2ccc(-n3c(=O)ccc4cnc5ccc(-c6cnc7ccccc7c6)cc5c43)cc2C(F)(F)F)CC1]

In some embodiments, the at least one mTOR inhibitor applicable for the invention is Torin-2 or at least one of solvate, a hydrate, a stereoisomer, a pharmaceutically acceptable prodrug, a pharmaceutically active metabolite, a pharmaceutically acceptable salt, a crystalline form, an amorphous form, a physiologically functional derivative of Torin-2 or any combinations thereof.

Torin-2 has a CAS number 1223001-51-1 and is represented by the following chemical structure represented by Formula (VIII):

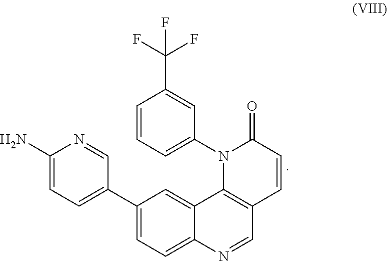 [SMILES: Nc1ccc(-c2ccc3ncc4ccc(=O)n(-c5cccc(C(F)(F)F)c5)c4c3c2)cn1]

In some embodiments, the at least one mTOR inhibitor applicable for the invention is vistusertib or at least one of solvate, a hydrate, a stereoisomer, a pharmaceutically acceptable prodrug, a pharmaceutically active metabolite, a pharmaceutically acceptable salt, a crystalline form, an amorphous form, a physiologically functional derivative of Vistusertib or any combinations thereof.

Vistusertib (AZD2014) has a CAS number 1009298-59-2 and is represented by the following chemical structure represented by Formula (IX):

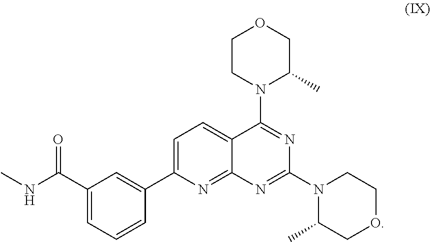 [SMILES: CNC(=O)c1cccc(-c2ccc3c(N4CCOC[C@@H]4C)nc(N4CCOC[C@@H]4C)nc3n2)c1]

In some embodiments, the mTOR inhibitor being a mTOR/PI3K dual inhibitor is at least one of dactolisib, voxtalisib, BGT226, SF1126, PKI-587, NVPBE235 or any combinations thereof.

In some embodiments, the mTOR inhibitor is at least one of dactolisib, voxtalisib. BGT226, SF1126, Gedatolisib, or any combinations thereof.

In some embodiments, the at least one mTOR inhibitor applicable for the invention is dactolisib or at least one of solvate, a hydrate, a stereoisomer, a pharmaceutically acceptable prodrug, a pharmaceutically active metabolite, a pharmaceutically acceptable salt, a crystalline form, an amorphous form, a physiologically functional derivative of dactolisib or any combinations thereof.

Dactolisib (codenamed NVP-BEZ235 and BEZ-235, also known as RTB101) has a CAS number 915019-65-7 and is represented by the following chemical structure represented by Formula (X):

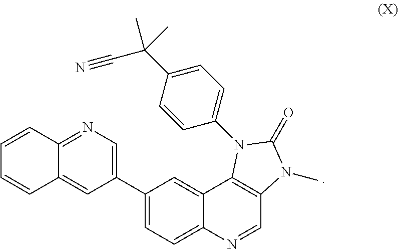 [SMILES: Cn1c(=O)n(-c2ccc(C(C)(C)C#N)cc2)c2c3cc(-c4cnc5ccccc5c4)ccc3ncc21]

In some embodiments, the at least one mTOR inhibitor applicable for the invention is voxtalisib at least one of solvate, a hydrate, a stereoisomer, a pharmaceutically acceptable prodrug, a pharmaceutically active metabolite, a pharmaceutically acceptable salt, a crystalline form, an amorphous form, a physiologically functional derivative of voxtalisib any combinations thereof.

Voxtalisib has a CAS number 934493-76-2 and is represented by the following chemical structure represented by Formula (XI):

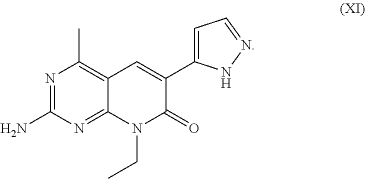 [SMILES: CCn1c(=O)c(-c2ccn[nH]2)cc2c(C)nc(N)nc21]

In some embodiments, the at least one mTOR inhibitor applicable for the invention is BGT226 at least one of solvate, a hydrate, a stereoisomer, a pharmaceutically acceptable prodrug, a pharmaceutically active metabolite, a pharmaceutically acceptable salt, a crystalline form, an amorphous form, a physiologically functional derivative of BGT226 any combinations thereof.

BGT226 (NVP-BGT226) maleate has a CAS number 1245537-68-1 and is represented by the following chemical structure represented by Formula (XII):

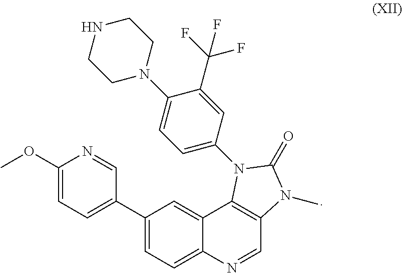

In some embodiments, the at least one mTbR inhibitor applicable for the invention is SF1126 at least one of solvate, a hydrate, a stereoisomer, a pharmaceutically acceptable prodrug, a pharmaceutically active metabolite, a pharmaceutically acceptable salt, a crystalline form, an amorphous form, a physiologically functional derivative of SF1126 any combinations thereof.

SF1126 has a CAS number 936487-67-1 and is represented by the following chemical structure represented by Formula (XIII):

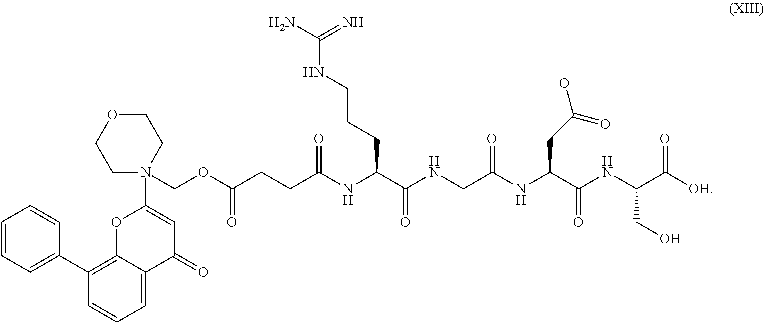

In some embodiments, the at least one mTOR inhibitor applicable for the invention is Gedatolisib at least one of solvate, a hydrate, a stereoisomer, a pharmaceutically acceptable prodrug, a pharmaceutically active metabolite, a pharmaceutically acceptable salt, a crystalline form, an amorphous form, a physiologically functional derivative of Gedatolisib any combinations thereof.

Gedatolisib (PKI-587) has a CAS number 1197160-78-3 and is represented by the following chemical structure represented by Formula (XIV):

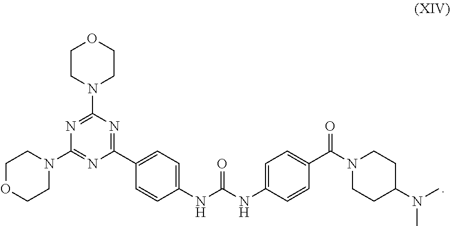 [SMILES: CN(C)C1CCN(C(=O)c2ccc(NC(=O)Nc3ccc(-c4nc(N5CCOCC5)nc(N5CCOCC5)n4)cc3)cc2)CC1]

In some embodiments, the mTOR inhibitor being a mTORC1/mTORC2 dual inhibitor is at least one of sapanisertib, AZD8055, and Vistusertibor any combinations thereof.

In some embodiments, the mTOR inhibitor is at least one of sapanisertib, AZD8055, and AZD2014, or any combinations thereof.

In some embodiments, the at least one mTOR inhibitor applicable for the invention is Sapanisertib at least one of solvate, a hydrate, a stereoisomer, a pharmaceutically acceptable prodrug, a pharmaceutically active metabolite, a pharmaceutically acceptable salt, a crystalline form, an amorphous form, a physiologically functional derivative of Sapanisertib any combinations thereof.

Sapanisertib (also known as MLN0128, INK128 and TAK-228) has a CAS number 1224844-38-5 and is represented by the following chemical structure represented by Formula (XV):

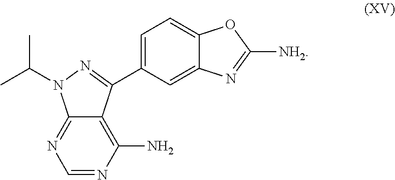 [SMILES: CC(C)n1nc(-c2ccc3oc(N)nc3c2)c2c(N)ncnc21]

In some embodiments, the at least one mTOR inhibitor applicable for the invention is AZD-8055 at least one of solvate, a hydrate, a stereoisomer, a pharmaceutically acceptable prodrug, a pharmaceutically active metabolite, a pharmaceutically acceptable salt, a crystalline form, an amorphous form, a physiologically functional derivative of AZD-8055 any combinations thereof.

AZD-8055 has a CAS number 1009298-09-2 and is represented by the following chemical structure represented by Formula (XVI):

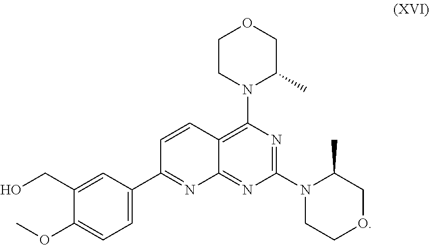 [SMILES: COc1ccc(-c2ccc3c(N4CCOC[C@@H]4C)nc(N4CCOC[C@@H]4C)nc3n2)cc1CO]

In some embodiments, the at least one mTOR inhibitor applicable for the invention is vistusertib or at least one of solvate, a hydrate, a stereoisomer, a pharmaceutically acceptable prodrug, a pharmaceutically active metabolite, a pharmaceutically acceptable salt, a crystalline form, an amorphous form, a physiologically functional derivative of vistusertib any combinations thereof.

Vistusertib (AZD2014 has a CAS number 1009298-59-2 and is represented by the following chemical structure represented by Formula (XVII):

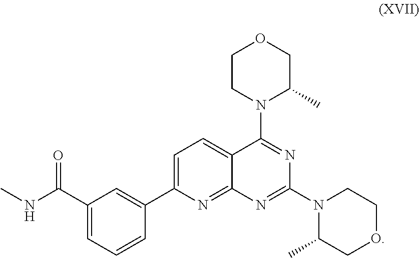 [SMILES: CNC(=O)c1cccc(-c2ccc3c(N4CCOC[C@@H]4C)nc(N4CCOC[C@@H]4C)nc3n2)c1]

In accordance with some aspects which can be implemented as embodiments of the invention, it is provided a combination comprising an inhibitor of mTOR and at least one modulator of an extrinsic oocyte pathway, wherein the at least one mTOR inhibitor being any one of rapamycin, temsirolimus, everolimus, ridaforolimus, umirolimus, zotarolimus, Torin-1, Torin-2, vistusertib, dactolisib, voxtalisib, BGT226, SF1126. PKI-587, NVPBE235, sapanisertib, AZD8055, and AZD2014, at least one of solvate, a hydrate, a stereoisomer, a pharmaceutically acceptable prodrug, a pharmaceutically active metabolite, a pharmaceutically acceptable salt, a crystalline form, an amorphous form, a physiologically functional derivative of at least one of any combinations thereof.

In accordance with some aspects which can be implemented as embodiments of the invention, it is provided a combination comprising an inhibitor of mTOR and at least one modulator of an extrinsic oocyte pathway, wherein the at least one mTOR inhibitor having any one of Formulas I, II, III, IV, V, VI, VII, VIII, IX, X, XI, XII, XIII, XIV, XV, XVI, XVII, at least one of solvate, a hydrate, a stereoisomer, a pharmaceutically acceptable prodrug, a pharmaceutically active metabolite, a pharmaceutically acceptable salt, a crystalline form, an amorphous form, a physiologically functional derivative of at least one of any combinations thereof.

In accordance with some aspects which can be implemented as embodiments of the invention, it is provided a combination comprising an inhibitor of mTOR and at least one modulator of an extrinsic oocyte pathway, wherein the at least one mTOR inhibitor having any one of Formulas I, II, III, at least one of solvate, a hydrate, a stereoisomer, a pharmaceutically acceptable prodrug, a pharmaceutically active metabolite, a pharmaceutically acceptable salt, a crystalline form, an amorphous form, a physiologically functional derivative of at least one of any combinations thereof.

In accordance with some aspects which can be implemented as embodiments of the invention, it is provided a combination comprising an inhibitor of mTOR and at least one modulator of an extrinsic oocyte pathway, wherein the at least one mTOR inhibitor having any one of Formulas I, II, III, IV, at least one of solvate, a hydrate, a stereoisomer, a pharmaceutically acceptable prodrug, a pharmaceutically active metabolite, a pharmaceutically acceptable salt, a crystalline form, an amorphous form, a physiologically functional derivative of at least one of any combinations thereof.

In accordance with some aspects which can be implemented as embodiments of the invention, it is provided a combination comprising an inhibitor of mTOR and at least one modulator of an extrinsic oocyte pathway, wherein the at least one mTOR inhibitor having any one of Formulas VII, VIII, IX, X, XI, XII, XIII, XIV, XV, XVI, XVII, at least one of solvate, a hydrate, a stereoisomer, a pharmaceutically acceptable prodrug, a pharmaceutically active metabolite, a pharmaceutically acceptable salt, a crystalline form, an amorphous form, a physiologically functional derivative of at least one of any combinations thereof.

The present disclosure also encompasses chemically modified compound derived from a parent small molecule of the invention (e.g., at least one mTOR inhibitor) that differs from the parent compound by one or more elements, substituents and/or functional groups such that the derivative has the same or similar biological properties/activities as the parent compound, as defined herein, specifically, in modulation of an intrinsic oocyte pathway.

Hence, the at least one mTOR inhibitor described herein having any one of Formulas I, II, III, IV, V, VI, VII, VIII, IX, X, XI, XII, XIII, XIV, XV, XVI, XVII may optionally be substituted with one or more substituents, thereby providing the derivatives of the invention.

As noted herein, the combination comprises a modulator of an extrinsic oocyte pathway.

As used herein, the term an extrinsic oocyte pathway refers to at least one substance that is secreted from a cell that is outside the oocyte or granulosa cells such as a neighboring cells or follicles. Such a pathway also encompasses physical forces acting on PMFs.

In some embodiments, the modulator of an extrinsic oocyte pathway is a member of the TGF-β superfamily.

As appreciated, the TGF-β superfamily refers to a large group of structurally related cell regulatory proteins which interact with TGF-β receptors, including type I receptors and type II receptors. These receptors include as type I receptors: Activin Receptor-like Kinase 1 (Alks1) receptor, Alks2 receptor, Alks2 receptor, Alks3 receptor, Alks4 receptor, Alks5 receptor, Alks6 receptor, and Alks7 receptor and as type II receptors: TGF-β type 2 (Tβ-R2) receptor, Bone Morphogenetic Protein Receptor Type 2 (BMPR2), Activin receptor type-2A (ActRIIA), activin receptor type-2B (ActRIIB), and anti-Mullerian hormone receptor type 2 (AMHR2).

In some embodiments, the modulator of an extrinsic oocyte pathway is capable of binding AMHR2 also known as Mullerian inhibiting substance type II receptor (MISIIR).

In the context of the present disclosure, the term AMHR2 or MISRII encompasses AMH receptor (AMHR2) or MIS receptor (MISIIR) as well as homologous of AMHR2 or MISRII and functional derivatives of AMHR2 or MISRII.

In some embodiments, the modulator of an extrinsic oocyte pathway is an agonist of AMHR2.

The term agonist as used herein refers to a chemical that activates a receptor to produce a biological response. Specifically, the agonist of the invention activates AMHR2. As noted herein, the agonist may be a direct agonist or an allosteric agonist (known for example as a positive allosteric modulators (PAM)).

In accordance with some aspects which can be implemented as embodiments of the invention, it is provided a combination comprising a modulator of an intrinsic oocyte pathway and at least one agonist of AMHR2.

In accordance with some aspects which can be implemented as embodiments of the invention, it is provided a combination comprising an inhibitor of mTOR and at least one agonist of AMHR2.

In accordance with some aspects which can be implemented as embodiments of the invention, it is provided a combination comprising an inhibitor of mTOR and at least one agonist of AMHR2, wherein the at least one mTOR inhibitor is at least one of rapamycin, temsirolimus, everolimus, ridaforolimus, umirolimus, zotarolimus, Torin-1, Torin-2, vistusertib, dactolisib, voxtalisib, BGT226, SF1126. PKI-587, NVPBE235, sapanisertib, AZD8055. and AZD2014, solvate, a hydrate, a stereoisomer, a pharmaceutically acceptable prodrug, a pharmaceutically active metabolite, a pharmaceutically acceptable salt, a crystalline form, an amorphous form, a physiologically functional derivative or any combinations thereof.

In some embodiments, the modulator of an extrinsic oocyte pathway being an agonist of AMHR2 is at least one of anti-mullerian hormone (AMH) protein.

As used herein anti-mullerian hormone (AMH) also known as Müllerian-inhibiting hormone (MIH) or Müllerian-inhibiting substance (MIS), is a dimeric glycoprotein with a molar mass of 140 kDa consisting of two identical subunits linked by sulfide bridges and characterized by the N-terminal dimer (pro-region) and C-terminal dimer (TGF-β domain).

In some embodiments, the AMH protein refers to the human AMH (hAMH).

In some embodiments the hAMH protein comprises an amino acid sequence of 560 amino acid residues as denoted by NCBI Accession No.: P03971. In some embodiments, the hAMH protein is or comprises an amino acid sequence as denoted by SEQ ID NO:1.

In some embodiments, the hAMH protein is or comprises the amino acid sequence:

MRDLPLTSLALVLSALGALLGTEALRAEEPAVGTSGLIFREDLDWPPGS
PQEPLCLVALGGDSNGSSSPLRVVGALSAYEQAFLGAVQRARWGPRDLA
TFGVCNTGDRQAALPSLRRLGAWLRDPGGQRLVVLHLEEVTWEPTPSLR
FQEPPPGGAGPPELALLVLYPGPGPEVTVTRAGLPGAQSLCPSRDTRYL
VLAVDRPAGAWRGSGLALTLQPRGEDSRLSTARLQALLFGDDHRCFTRM
TPALLLLPRSEPAPLPAHGQLDTVPFPPPRPSAELEESPPSADPFLETL
TRLVRALRVPPARASAPRLALDPDALAGFPQGLVNLSDPAALERLLDGE
EPLLLLLRPTAATTGDPAPLHDPTSAPWATALARRVAAELQAAAAELRS
LPGLPPATAPLLARLLALCPGGPGGLGDPLRALLLLKALQGLRVEWRGR
DPRGPGRAQRSAGATAADGPCALRELSVDLRAERSVLIPETYQANNCQG
VCGWPQSDRNPRYGNHVVLLLKMQVRGAALARPPCCVPTAYAGKLLISL
SEERISAHHVPNMVATECGCR.

In some other embodiments, hAMH protein is encoded by a nucleic acid sequence denoted by NCBI Reference Sequence: NM_000479. In some specific embodiments, hAMH protein is encoded by a nucleic acid sequence provided herein below by SEQ ID NO:2.

In the context of the present disclosure, when referring to AMH protein it may encompass a derivative, an analogue, a variant or a homologue of the AMH protein, specifically hAMH.

In addition, the present disclosure encompasses a derivative of AMH analogue, AMH variant or AMH homologue, specifically a derivative of hAMH analogue, hAMH variant or hAMH homologue.

In the context of the present disclosure, a derivative, an analogue, a variant or a homologue of the AMH protein or a derivative of AMH analogue, AMH variant or AMH homologue are considered to have the same or similar biological properties/activities as the AMH, specifically hAMH, when used in the combination therapy of the invention, to retain PMF reserve and increase fertility.

It should be also noted that when referring to a derivative, an analogue, a variant or a homologue, the same modification applies to the nucleic acid sequence resulting in a derivative, an analogue, a variant or a homologue of the protein, specifically hAMH, including corresponding substitution, deletion or insertion of corresponding codons.

The AMH protein may be isolated and purified by methods known in the art. Purification can be achieved by protein purification procedures such as chromatography methods (gel-filtration, ion-exchange and immunoaffinity), by high-performance liquid chromatography (such as HPLC) or by precipitation (immunoprecipitation).

Alternatively, AMH may be produced synthetically, or by recombinant DNA technology. Methods for producing polypeptides peptides are well known in the art.

In accordance with some aspects which can be implemented as embodiments of the invention, it is provided a combination comprising a modulator of an intrinsic oocyte pathway and AMH, a derivative, an analogue, a variant, or a homologue thereof.

In accordance with some aspects which can be implemented as embodiments of the invention, it is provided a combination comprising (i) at least one mTOR inhibitor and (ii) hAMH, a derivative, an analogue, a variant, or a homologue thereof.

In accordance with some aspects which can be implemented as embodiments of the invention, it is provided a combination comprising (i) at least one mTOR inhibitor and (ii) hAMH, a derivative, an analogue, a variant, or a homologue thereof, wherein the at least one mTOR inhibitor is at least one of rapamycin, temsirolimus, everolimus, ridaforolimus, umirolimus, zotarolimus, Torin-1, Torin-2. vistusertib, dactolisib, voxtalisib, BGT226, SF1126, PKI-587, NVPBE235, sapanisertib, AZD8055, and AZD2014. solvate, a hydrate, a stereoisomer, a pharmaceutically acceptable prodrug, a pharmaceutically active metabolite, a pharmaceutically acceptable salt, a crystalline form, an amorphous form, a physiologically functional derivative or any combinations thereof.

In some embodiments, the hAMH is an isolated and purified hAMH.

In some other embodiments, the AMH is a recombinant hAMH (rAMH).

In some embodiments, an homologue of the AMH protein comprises a protein (polypeptide, an amino acid sequence) having at least 60%, at least 65%, at least 70%, at least 75%, at least 80%, at least 85%, at least 90%, at least 95%, at least 97% identity to an amino acid sequence denoted by SEQ ID NO:1.

In some embodiments, an homologue of the AMH protein comprises an amino acid sequence that has between 40% to 99%, at times between 60% and 99%, at times between 90% to 99%, at times between 95% to 99% identity to an amino acid sequence denoted by SEQ ID NO:1.

In some embodiments, an homologue of the AMH protein comprises an amino acid sequence having 60%, 65%, 70%, 75%, 80%, %, 90%, 95%, 97% identity to an amino acid sequence denoted by SEQ ID NO:1.

In some embodiments, an homologue of the AMH protein comprises a protein (polypeptide, an amino acid sequence) encoded by a nucleic acid sequence having at least 60%, at least 65%, at least 70%, at least 75%, at least 80%, at least 85%, at least 90%, at least 95%, at least 97% identity to a nucleic acid sequence denoted by SEQ ID NO:2.

In some embodiments, an homologue of the AMH protein is encoded by a nucleic acid sequence that has between 40% to 99%, at times between 60% and 99%, at times between 90% to 99%, at times between 95% to 99% identity to a nucleic acid sequence denoted by SEQ ID NO:2.

In some embodiments, an homologue of the AMH protein is encoded by a nucleic acid sequence that has 60%, 65%, 70%, 75%, 80%, %, 90%, 95%, 97% identity to a nucleic acid sequence denoted by SEQ ID NO:2.

The % identity between two or more amino acid sequences is determined for the two or more sequences when compared and aligned for maximum correspondence. In the context of the present disclosure, sequences (amino acid or nucleic acid) as described herein having % identity are considered to have the same function/activity of the reference sequence to which identity is calculated to.

In some embodiments, an homologue of the AMH protein comprises an amino acid sequence having at least 60%, at least 65%, at least 70%, at least 75%, at least 80%, at least 85%, at least 90%, at least 95%, at least 97% similarity to an amino acid sequence denoted by SEQ ID NO:1.

In some embodiments, an homologue of the AMH protein comprises an amino acid sequence that has between 40% to 99%, at times between 60% and 99%, at times between 90% to 99%, at times between 95% to 99% similarity to an amino acid sequence denoted by SEQ ID NO:1.

In some embodiments, an homologue of the AMH protein comprises an amino acid sequence having 60%, 65%, 70%, 75%, 80%, %, 90%, 95%, 97% similarity to an amino acid sequence denoted by SEQ ID NO:1.

In some embodiments, an homologue of the AMH protein comprises a protein (polypeptide, an amino acid sequence) encoded by a nucleic acid sequence having at least 60%, at least 65%, at least 70%, at least 75%, at least 80%, at least 85%, at least 90%, at least 95%, at least 97% similarity to a nucleic acid sequence denoted by SEQ ID NO:2.

In some embodiments, an homologue of the AMH protein is encoded by a nucleic acid sequence that has between 40% to 99%, at times between 60% and 99%, at times between 90% to 99%, at times between 95% to 99% similarity to a nucleic acid sequence denoted by SEQ ID NO:2.

In some embodiments, an homologue of the AMH protein is encoded by a nucleic acid sequence that has 60%, 65%, 70%, 75%, 80%, %, 90%, 95%, 97% similarity to a nucleic acid sequence denoted by SEQ ID NO:2.

In Sequence similarity or sequence homology as used herein refers to the amount (%) of amino acids that conserved with similar physicochemical properties, for example in considering amino acid similarity, for example leucine and isoleucine.

In determining the sequence identity, the gaps are typically not counted, and the sequence identity is relative to the shorter sequence of the two. In this connection, it should be noted that the length for example of the AMH protein (amino acid sequence) may be the same as the homologue protein (amino acid sequence) or may be different than the homologue protein (amino acid sequence).

The term “amino acid sequence” and/or “polypeptide” are used to describe a protein having an amino acid sequence or polypeptide chain connected by peptide bonds. A polypeptide sequence is generally reported from the N-terminal end containing free amino group to the C-terminal end containing free carboxyl group.

Amino acid sequence is often called peptide, protein sequence if it represents the primary structure of a protein whereas protein refers to as an amino acid sequence folded into a specific three-dimensional configuration. This three-dimensional configuration can typically undergo post-translational modifications.

The terms “amino acid sequence” and/or “polypeptide” may refer to sequences having a 3D structure (protein) as well as sequences with no 3D structure.

As described herein, the present disclosure encompasses any fragments (peptides) derived from AMH, specifically of hAMH.

According to some embodiments, a fragment of AMH protein may comprise the TGF-β domain of AMH.

In some embodiments, the fragment of AMH comprises at least the C-terminal end of AMH (“at times referred herein as C′-terminus domain”).

In some embodiments, the fragment of AMH comprises an amino acid sequence comprising between 15 to 120 amino acids, at times 20 to 120, at times 30 to 120, at times 40 to 120, at times 50 to 120, at times 60 to 120, at times 70 to 120, at times 80 to 120, at times 90 to 120, at times 99 to 120, at times 99 to 115 amino acid residues at least the C-terminal end of AMH.

In some embodiments, the AMH fragment comprises an amino acid sequence of 99 amino acid residues at the C′-terminus domain of AMH.

In some embodiments, the hAMH fragment comprises an amino acid sequence between amino acid residue 462 to amino acid residue 560 in SEQ ID NO:1.

In some embodiments the hAMH fragment comprises an amino acid sequence CALRELSVDLRAERSVLIPETYQANNCQGVCGWPQSDRNPRYGNHVVLLLKM QVRGAALARPPCCVPTAYAGKLLISLSEERISAHHVPNMVATECGCR.

In some embodiments the amino acid sequence CALRELSVDLRAERSVLIPETYQANNCQGVCGWPQSDRNPRYGNHVVLLLKM QVRGAALARPPCCVPTAYAGKLLISLSEERISAHHVPNMVATECGCR is denoted by SEQ ID NO:3.

In some embodiments, the hAMH fragment comprises an amino acid sequence denoted by any one of SEQ ID NO: 3.

In some embodiments, the AMH fragment consists of an amino-acid sequence as denoted by any one of SEQ ID NO: 3.

In some embodiments, the AMH fragment comprises an amino acid sequence of 109 amino acid residues at the C′-terminus domain of AMH.

In some embodiments, the AMH fragment comprises an amino acid sequence between amino acid residue 452 to amino acid residue 560 in SEQ ID NO:1.

In some embodiments the AMH fragment comprises an amino acid sequence of SAGATAADGPCALRELSVDLRAERSVLIPETYQANNCQGVCGWPQSDRNPRYG NHVVLLLKMQVRGAALARPPCCVPTAYAGKLLISLSEERISAHHVPNMVATEC GCR.

In some embodiments the amino acid sequence SAGATAADGPCALRELSVDLRAERSVLIPETYQANNCQGVCGWPQSDRNPRYG NHVVLLLKMQVRGAALARPPCCVPTAYAGKLLISLSEERISAHHVPNMVATEC GCR is denoted by SEQ ID NO: 4.

In some embodiments, the hAMH fragment comprises an amino-acid sequence as denoted by SEQ ID NO: 4.

In some embodiments, the hAMH fragment consists of an amino-acid sequence as denoted by SEQ ID NO: 4.

In some embodiments, the AMH fragment comprises an amino acid sequence of 110 amino acid residues at the C′-terminus domain of AMH.

In some embodiments, the AMH fragment comprises an amino acid sequence between amino acid residue 451 to amino acid residue 560 in SEQ ID NO:1.

In some embodiments the AMH fragment comprises an amino acid sequence RSAGATAADGPCALRELSVDLRAERSVLIPETYQANNCQGVCGWPQSDRNPRY GNHVVLLLKMQVRGAALARPPCCVPTAYAGKLLISLSEERISAHHVPNMVATE CGCR.

In some embodiments the amino acid sequence RSAGATAADGPCALRELSVDLRAERSVLIPETYQANNCQGVCGWPQSDRNPRY GNHVVLLLKMQVRGAALARPPCCVPTAYAGKLLISLSEERISAHHVPNMVATE CGCR is denoted by SEQ ID NO:5.

In some embodiments, the hAMH fragment comprises an amino-acid sequence as denoted by a SEQ ID NO:5.

In some embodiments, the hAMH fragment consists of an amino-acid sequence as denoted by SEQ ID NO: 5.

In some embodiments, the hAMH fragment comprises an amino acid sequence of 108 amino acid residues at the C′-terminus domain of AMH.

In some embodiments, the AMH fragment comprises an amino acid sequence between amino acid residue 453 to amino acid residue 560 in SEQ ID NO:1.

In some embodiments the AMH fragment comprises an amino acid sequence AGATAADGPCALRELSVDLRAERSVLIPETYQANNCQGVCGWPQSDRNPRYG NHVVLLLKMQVRGAALARPPCCVPTAYAGKLLISLSEERISAHHVPNMVATEC GCR.

In some embodiments the amino acid sequence AGATAADGPCALRELSVDLRAERSVLIPETYQANNCQGVCGWPQSDRNPRYG NHVVLLLKMQVRGAALARPPCCVPTAYAGKLLISLSEERISAHHVPNMVATEC GCR is denoted by SEQ ID NO:6 In some embodiments, the AMH fragment comprises an amino-acid sequence as denoted by SEQ ID NO:6.

In some embodiments, the AMH fragment consists of an amino-acid sequence as denoted by SEQ ID NO: 6.

In some embodiments, the hAMH fragment comprises an amino acid sequence denoted by at least one of SEQ ID NO:3, SEQ ID NO:4, SEQ ID NO:5, or SEQ ID NO:6.

In some embodiments, the hAMH fragment comprises an amino acid sequence denoted by at least one of SEQ ID NO:4, SEQ ID NO:5, or SEQ ID NO:6.

In accordance with some aspects which can be implemented as embodiments of the invention, it is provided a combination comprising (i) at least one mTOR inhibitor and (ii) a polypeptide having an amino acid sequence denoted by at least one of SEQ ID NO:1, SEQ ID NO:3, SEQ ID NO:4, SEQ ID NO:5, or SEQ ID NO:6, a derivative, an analogue, a variant, or a homologue thereof.

In accordance with some aspects which can be implemented as embodiments of the invention, it is provided a combination comprising (i) at least one mTOR inhibitor and (ii) a polypeptide having an amino acid sequence denoted by at least one of SEQ ID NO:4, SEQ ID NO:5, or SEQ ID NO:6, a derivative, an analogue, a variant, or a homologue thereof.

In accordance with some aspects which can be implemented as embodiments of the invention, it is provided a combination comprising (i) at least one mTOR inhibitor and (ii) a polypeptide having an amino acid sequence denoted by at least one of SEQ ID NO:1, SEQ ID NO:3, SEQ ID NO:4, SEQ ID NO:5, or SEQ ID NO:6, a derivative, an analogue, a variant, or a homologue thereof, wherein the at least one mTOR inhibitor is at least one of rapamycin, temsirolimus, everolimus, ridaforolimus, umirolimus, zotarolimus, Torin-1, Torin-2. vistusertib, dactolisib, voxtalisib, BGT226, SF1126, PKI-587. NVPBE235, sapanisertib, AZD8055. and AZD2014. solvate, a hydrate, a stereoisomer, a pharmaceutically acceptable prodrug, a pharmaceutically active metabolite, a pharmaceutically acceptable salt, a crystalline form, an amorphous form, a physiologically functional derivative or any combinations thereof.

In addition, and as noted herein, the present disclosure encompasses any derivative, analogue, variant, or homologue of any fragments (peptides) derived from hAMH. In other words, the present disclosure encompasses fragments and peptides of hAMH derivatives as well as homologous sequence of fragments or peptides thereof.

In some embodiments, a homologue of a fragment of the AMH sequence comprises an amino acid sequence having at least 60%, at least 65%, at least 70%, at least 75%, at least 80%, at least 85%, at least 90%, at least 95%, at least 97% identity to an amino acid sequence being at least one of SEQ ID NO:3, SEQ ID NO:4, SEQ ID NO:5, or SEQ ID NO:6.

In some embodiments, a homologue of a fragment of the AMH sequence comprises an amino acid sequence having between 40% to 99%, at times between 60% and 99%, at times between 90% to 99%, at times between 95% to 99% identity to an amino acid sequence being at least one of SEQ ID NO:3, SEQ ID NO:4, SEQ ID NO:5, or SEQ ID NO:6.

In some embodiments, a homologue of a fragment of the AMH sequence comprises an amino acid sequence having 60%, 65%, 70%, 75%, 80%, %, 90%, 95%, 97% identity to an amino acid sequence being at least one of SEQ ID NO:3, SEQ ID NO:4, SEQ ID NO:5, or SEQ ID NO:6.

In some embodiments, a homologue of a fragment of the AMH sequence comprises an amino acid sequence having at least 60%, at least 65%, at least 70%, at least 75%, at least 80%, at least 85%, at least 90%, at least 95%, at least 97% similarity to an amino acid sequence being at least one of SEQ ID NO:3, SEQ ID NO:4, SEQ ID NO:5, or SEQ ID NO:6.

In some embodiments, a homologue of a fragment of the AMH sequence comprises an amino acid sequence having between 40% to 99%, at times between 60% and 99%, at times between 90% to 99%, at times between 95% to 99% similarity to an amino acid sequence being at least one of SEQ ID NO:3, SEQ ID NO:4, SEQ ID NO:5, or SEQ ID NO:6.

In some embodiments, a homologue of a fragment of the AMH sequence comprises an amino acid sequence having 60%, 65%, 70%, 75%, 80%, %, 90%, 95%, 97% similarity to an amino acid sequence being at least one of SEQ ID NO:3, SEQ ID NO:4, SEQ ID NO:5, or SEQ ID NO:6.

In accordance with some aspects which can be implemented as embodiments of the invention, it is provided a combination comprising (i) at least one mTOR inhibitor and (ii) a polypeptide denoted by at least one of SEQ ID NO:1, SEQ ID NO:3, SEQ ID NO:4, SEQ ID NO:5 or SEQ ID NO:6. In some embodiments, the at least one mTOR inhibitor is at least one of Formulas I, II, III, IV, V, VI, VII, VIII, IX, X, XI, XII, XIII, XIV, XV, XVI, XVII or any combination thereof.

As described herein and shown in the Examples below, the combinations of the invention were effective in retaining (maintaining) PMF reserve.

The term “retaining” as used herein refers to maintaining or keeping the amount and quality of the ovarian follicular reserve.

Hence, in accordance with some aspects which can be implemented as embodiments of the invention, it is provided a combination for use in retaining PMF reserve.

“PMF reserve” as used herein refers to the number and quality of primordial follicles in the ovary at a given age of a subject. As appreciated, PMF reserve is ultimately depleted by degeneration and progression until exhausted.

In some embodiments, the combination of the invention is for use for retaining and maintaining the number as well as the quality of ovarian follicles, increasing viability and functionality and preventing apoptosis and death of the ovarian follicles, specifically PMF. As noted herein, this may result in increased fertilization rate and implementation rate.

Retaining PMF reserve may be in accordance with the present disclosure by inhibiting at least one of primordial follicles activation, ovarian follicles depletion, premature ovarian insufficiency (POI), destruction of the oocyte pool, follicle apoptosis or any combination thereof.

In accordance with some aspects which may be considered as embodiments of the invention, the combination of the invention is for use in inhibiting primordial follicle activation.

As used herein “primordial follicle activation” as used herein refer to excessive induction of PMF activation that may cause development of premature ovarian insufficiency (POI).

Premature ovarian insufficiency (POI) is characterized by decreased ovarian follicles that accelerate the onset of menopause and results in subfertility or infertility, causing menstrual irregularities and pregnancy failures POI may results for example from early follicular depletion, blockage of follicular maturation or destruction of the oocyte pool.

In accordance with some aspects which may be considered as embodiments of the invention, the combination of the invention is for use in inhibiting POI.

In accordance with some aspects which may be considered as embodiments of the invention, the combination of the invention is for use in inhibiting follicle apoptosis.

As used herein inhibiting, preventing or reducing at least one of primordial follicles activation, ovarian follicles depletion, premature ovarian insufficiency (POI), destruction of the oocyte pool, follicle apoptosis or any combination thereof, maintain (retain, keep) at least part of the follicles in their primordial stage, by inhibiting early follicular depletion.

As appreciated, inhibiting, preventing or reducing at least one of primordial follicles activation, ovarian follicles depletion, premature ovarian insufficiency (POI), destruction of the oocyte pool, follicle apoptosis or any combination thereof may in accordance with some embodiments to increase fertility in a subject.

As used herein the term increase fertility refers to an increase in is the ability of a subject to produce offspring through reproduction following the onset of sexual maturity.

Hence, in accordance with some aspects which may be considered as embodiments of the invention, the combination of the invention is for use in increasing fertility in a subject.

In accordance with some aspects, it is provided a combination of the invention for use in a method of retaining PMF in a subject.

In accordance with some aspects, it is provided a combination of the invention for use in a method of reducing, decreasing, inhibiting or preventing at least one of primordial follicles activation, ovarian follicles depletion, POI, destruction of the oocyte pool, follicle apoptosis or any combination thereof in a subject.

In accordance with some aspects, it is provided a combination of the invention for use in a method of reducing, decreasing, inhibiting or preventing POI in a subject.

In accordance with some aspects, it is provided a combination of the invention for use in a method of increasing fertility in a subject.

It should be noted that in accordance with the present disclosure increasing fertility in a subject refers to treating, inhibiting, preventing or ameliorating infertility or related conditions in the subject.

Infertility as used herein refers to a woman who is unable to conceive as well as being unable to carry a pregnancy to full term. As described herein, the infertility is caused by the condition of the subject being age, genetic cause or a non-genetic cause.

In accordance with some aspects which can be implemented as embodiments of the invention, it is provided a combination comprising (i) an inhibitor of the PI3K pathway and (ii) hAMH or fragments thereof for use in at least one of retaining PMF, inhibiting POI, increasing fertility or a combination thereof in a subject.

In accordance with some aspects which can be implemented as embodiments of the invention, it is provided a combination comprising (i) an inhibitor of the PI3K pathway and (ii) a polypeptide denoted by at least one of SEQ ID NO:1, SEQ ID NO:3, SEQ ID NO:4, SEQ ID NO:5 or SEQ ID NO:6 for use in at least one of retaining PMF, inhibiting POI, increasing fertility or a combination thereof in a subject.

In accordance with some aspects which can be implemented as embodiments of the invention, it is provided a combination comprising (i) at least one mTOR inhibitor and (ii) hAMH, or fragments thereof for use in at least one of retaining PMF, inhibiting POI, increasing fertility or a combination thereof in a subject.

In accordance with some aspects which can be implemented as embodiments of the invention, it is provided a combination comprising (i) at least one mTOR inhibitor being at least one of Formulas I, II, III, IV, V, VI, VII, VIII, IX, X, XI, XII, XIII, XIV, XV, XVI, XVII or any combination thereof and (ii) hAMH, or fragments thereof for use in at least one of retaining PMF, inhibiting POI, increasing fertility or a combination thereof in a subject.

In accordance with some aspects which can be implemented as embodiments of the invention, it is provided a combination comprising (i) at least one mTOR inhibitor being at least one of Formulas I, II, III, or any combination thereof and (ii) hAMH, or fragments thereof for use in at least one of retaining PMF, inhibiting POI, increasing fertility or a combination thereof in a subject.

In accordance with some aspects which can be implemented as embodiments of the invention, it is provided a combination comprising (i) at least one mTOR inhibitor and (ii) a polypeptide denoted by at least one of SEQ ID NO:1, SEQ ID NO:3, SEQ ID NO:4, SEQ ID NO:5 or SEQ ID NO:6 for use in at least one of retaining PMF, inhibiting POI, increasing fertility or a combination thereof in a subject.

In accordance with some aspects which can be implemented as embodiments of the invention, it is provided a combination comprising (i) at least one mTOR inhibitor being at least one of Formulas I, II, III, IV, V, VI, VII, VIII, IX, X, XI, XII, XIII, XIV, XV, XVI, XVII or any combination thereof and (ii) a polypeptide denoted by at least one of SEQ ID NO:1, SEQ ID NO:3, SEQ ID NO:4, SEQ ID NO:5 or SEQ ID NO:6 for use in at least one of retaining PMF, inhibiting POI, increasing fertility or a combination thereof in a subject.

In accordance with some aspects which can be implemented as embodiments of the invention, it is provided a combination comprising (i) at least one mTOR inhibitor being at least one of Formulas I, II, III, or any combination thereof and (ii) a polypeptide denoted by at least one of SEQ ID NO:1, SEQ ID NO:3, SEQ ID NO:4, SEQ ID NO:5 or SEQ ID NO:6 for use in at least one of retaining PMF, inhibiting POI, increasing fertility or a combination thereof in a subject.

Thus, in yet a further aspect, the invention relates to a method of retaining PMF in a subject, the method comprises the step of administering to the subject a therapeutically effective amount of the combination of the invention.

In yet a further aspect, the invention relates to a method of inhibiting or reducing POI in a subject, the method comprises the step of administering to the subject a therapeutically effective amount of the combination of the invention.

Further, in accordance with some other aspects, the present disclosure provides a method of increasing fertility in a subject, the method comprises the step of administering to the subject a therapeutically effective amount of the combinations of the invention.

In some embodiments, the subject is a female subject.

In some embodiments, the female subject refers to women and adolescent children.

In some embodiments, the subject is a female subject in need to retain PMF. In some embodiments, the subject is a female subject in need to increase fertility.

In accordance with some aspects which can be implemented as embodiments of the invention, the combination implemented in the methods of the invention may comprise (i) at least one mTOR inhibitor and (ii) hAMH or fragments thereof.

In accordance with some aspects which can be implemented as embodiments of the invention, the combination implemented in the methods of the invention may comprise (i) at least one mTOR inhibitor and (ii) a polypeptide denoted by at least one of SEQ ID NO:1, SEQ ID NO:3, SEQ ID NO:4, SEQ ID NO:5 or SEQ ID NO:6.

In accordance with some aspects which can be implemented as embodiments of the invention, the combination implemented in the methods of the invention may comprise (i) at least one mTOR inhibitor being at least one of Formulas I, II, III, IV, V, VI, VII, VIII, IX, X, XI, XII, XIII, XIV, XV, XVI, XVII or any combination thereof and (ii) hAMH, or fragments thereof.

In accordance with some aspects which can be implemented as embodiments of the invention, the combination implemented in the methods of the invention may comprise (i) at least one mTOR inhibitor being at least one of Formulas I, II, III, IV, V, VI, VII, VIII, IX, X, XI, XII, XIII, XIV, XV, XVI, XVII or any combination thereof and (ii) a polypeptide denoted by at least one of SEQ ID NO:1, SEQ ID NO:3, SEQ ID NO:4, SEQ ID NO:5 or SEQ ID NO:6.

In some embodiments, methods and uses of the invention employ administering to a subject in need to retain PMF and/or to increase fertility the combinations of the invention, wherein the subject is suffering from at least one of ovarian follicles depletion, PMF depletion, apoptosis and death of the ovarian follicles, primordial follicles activation, premature ovarian insufficiency (POI), destruction of the oocyte pool, reduced infertility or any combination thereof.

In some embodiments, the female subject is in need to delay pregnancy, or a female subject in need become pregnant at a later stage as well as to delay childbearing years to a later time point.

In some embodiments, the female subject may be at any age ranging between 17-50 years, 17-25, 25-30, 30-35, 35-40, 40-45, 45-50 or older than 50-years.

In some embodiments, the female subject is in the reproductive years. The term reproductive years refers in general to the age prior to menopause.

In some embodiments, the subject is a female subject suffering from primordial follicles activation.

In some embodiments, the subject is a female subject suffering from POI.

In some embodiments, the subject is a female subject suffering from reduced fertility.

In some embodiments, the subject is a female subject suffering from reduced fertility due to premature follicles activation and POI.

In some embodiments, the POI is a result of a genetic cause.

In some embodiments, the POI is a result genetic abnormality. In some embodiments, the POI is associated with an abnormality in at least one gene.

In some embodiments, the premature ovarian insufficiency is associated with an abnormality in BReast CAncer gene 1 (BRCA1) and/or BReast CAncer gene 2 (BRCA2).

In some embodiments, the premature ovarian insufficiency is associated with Turner syndrome (TS) (also known as 45, X, or 45, X0).

In some embodiments, the premature ovarian insufficiency is associated with polycystic ovary syndrome (PCOS).

In some embodiments, the POI is associated with resistant ovary syndrome.

In some embodiments, the POI is a result of a non-genetic cause.

In some embodiments, the non-genetic cause is at least one of an autoimmune disorder, a metabolic disorder, an infection, an environmental factor, a medical procedure, or any combinations thereof.

In some embodiment, the primordial follicle activation and/or POI is induced by a medical procedure.

In some embodiment, the POI is induced by a medical procedure.

In some embodiments, the subject is a female subject undergoing a medical procedure with an agent that induces primordial follicle activation/POI and hence follicle depletion (loss).

In some embodiments, the medical procedure is an anti-cancer treatment.

In some embodiments, the anti-caner treatment results in at least one of accelerated primordial follicle activation, ovarian follicles depletion, destruction of the oocyte pool or any combination thereof.

In some embodiments, the anti-cancer treatment is at least one of chemotherapy, radiotherapy or a combination thereof.

In some embodiments, the anti-cancer treatment is chemotherapy. The side effects of chemotherapy may include short-term and long-term effects and are often associated with destruction of ovarian follicle reserve.

In some embodiments, the invention is applicable to female subject being treated or was treated with an anti-cancer treatment.

In some embodiments, the invention is applicable to female subject undergoing treatment with chemotherapy or was treated with chemotherapy.

The present invention is not related to a specific chemotherapeutic agent and is applicable to various chemotherapeutic agents that may have one of the following: induce apoptosis of the primordial follicle pool, induce loss of PMF and reduced fertility.

In some embodiments, the chemotherapy is at least one of at least one alkylating agent, at least one anti metabolite, at least one anti-tumor antibiotics, at least one Topoisomerase inhibitor, at least one mitotic inhibitor, at least one plant alkaloid or a combination thereof.

In some embodiments, the chemotherapy is at least one alkylating agent. As appreciated, an alkylating agent attaches an alkyl group to DNA, specifically to the guanine base of DNA.

An alkylating agent as used herein refers to a compound that attaches an alkyl group to DNA.

In some embodiments, the at least one alkylating-like agent is at least one of a nitrogen mustard, a nitrosourea, an alkyl sulfonate.

In some embodiments, the nitrogen mustard is at least one of cyclophosphamide, chlormethine, uramustine, melphalan, chlorambucil, ifosfamide, bendamustine or any combination thereof.

In some embodiments, the chemotherapy is cyclophosphamide (CP, Cy) or any metabolite thereof.

In some embodiments, the nitrosourea is at least one of carmustine, lomustine, streptozocin or any combination thereof.

In some embodiments, the alkyl sulfonate is busulfan.

In some embodiments, the chemotherapy is at least one alkylating-like agent.

The alkylating-like agent do not have an alkyl group, but damage DNA by permanently coordinate to DNA to interfere with DNA repair and are sometimes described as “alkylating-like” agent.

In some embodiments, the at least one alkylating-like agent is at least one platinum-based agent.

In some embodiments, the at least one platinum-based agent is at least one of cisplatin, carboplatin, dicycloplatin, eptaplatin, lobaplatin, miriplatin, nedaplatin, oxaliplatin, picoplatin, satraplatin or combinations thereof.

In some embodiments, the anti-cancer treatment is radiotherapy. In other embodiments, the subject is a female subject undergoing treatment with radiotherapy or was treated with radiotherapy.

In some embodiments, the treatment is a gonadotoxic treatment. As used herein the term gonadotoxic treatment refers chemotherapy, radiation, or surgical resection (for treatment of disease or gender affirmation surgery).

In the context of the present disclosure the gonadotoxic treatment is capable of causing one or more of ovarian follicles depletion, PMF depletion, apoptosis and death of the ovarian follicles, primordial follicles activation, premature ovarian insufficiency (POI), destruction of the oocyte pool, reduced infertility or any combination thereof.

In some embodiments, the female subject is diagnosed with a cancer.

In some embodiments, the female subject is treated or was treated with anti-cancer treatment as described herein.

In some embodiments, the female subject is diagnosed with a cancer and is treated or was treated with anti-cancer treatment.

In some embodiments, the female subject is diagnosed with a cancer and is treated or was treated with anti-cancer treatment and is suffering from at least one of ovarian follicles depletion, PMF depletion, apoptosis and death of the ovarian follicles, primordial follicles activation, premature ovarian insufficiency (POI), destruction of the oocyte pool, reduced infertility or any combination thereof.

Cancer as defined herein is a disorder displaying cell division and growth that is not part of normal cellular turnover, metabolism, growth, or propagation of the whole organism. Unwanted proliferation of cells is seen in tumors and other pathological proliferation of cells, does not serve normal function, and for the most part will continue unbridled at a growth rate exceeding that of cells of a normal tissue in the absence of outside intervention. A pathological state that ensues because of the unwanted proliferation of cells is referred herein as a “hyper-proliferative disease” or “hyper-proliferative disorder.” It should be noted that the term “proliferative disorder”, “cancer”, “tumor” and “malignancy” all relate equivalently to a hyperplasia of a tissue or organ.

It should be noted that the term cancer when used herein encompasses non-invasive cancer, invasive cancer and metastatic cancer.

“Non-invasive” cancer is to be understood as a cancer that do not grow into or invade normal tissues within or beyond the primary location, for example the ovary.

“Invasive cancers” is to be understood as cancer that invades and grows in normal, healthy tissues to form metastasis.

“Metastatic cancer” or “metastatic status” is to be understood as a cancer that has spread from the place where it first started to another place in the body. Such a tumor formed by metastatic cancer cells is called a metastatic tumor or a metastasis.

Malignancy encompasses any one of carcinomas, melanomas, lymphomas, leukemias, myeloma and sarcomas.

Malignancies of tissues or organs may produce solid tumors. If the tissue is a part of the lymphatic or immune systems, malignant cells may include non-solid tumors of circulating cells. In general, the methods of the present invention may be applicable for patients suffering of non-solid tumors as well as of solid tumors.

In some embodiments, the cancer is selected from the group consisting of breast cancer, renal cancer, melanoma, lung cancer, glioblastoma, head and neck cancer, prostate cancer, ovarian carcinoma, bladder carcinoma, primary peritoneal carcinomatosis, genitourinary cancer, metastatic peritoneal carcinomatosis, and lymphoma.

In some embodiments, the cancer may be any one of leukemias and lymphoma.

In some embodiments, the cancer may be any one of carcinoma, melanoma, sarcoma, glioma and blastoma.

In some embodiments, the cancer is a carcinoma. In some embodiments, the carcinoma is an adenocarcinoma, a basal cell carcinoma, or squamous cell carcinoma.

In some embodiments, the cancer is selected from the group consisting of brain cancer, breast cancer, and lung cancer.

In some embodiments, the methods and uses of the invention employ administering to a subject suffering from at least one of ovarian follicles depletion, PMF depletion, apoptosis and death of the ovarian follicles, primordial follicles activation, premature ovarian insufficiency (POI), destruction of the oocyte pool, reduced infertility or any combination thereof a therapeutically effective amount of a combination of the invention.

In some embodiments, methods and uses of the invention employ administering to a subject in need to retain PMF and/or to increase fertility the combinations of the invention, wherein the subject is suffering from caner and is undergoing treatment with chemotherapy or was treated with chemotherapy.

In some embodiments, the methods and uses of the invention employ administering to a subject a combination comprising (i) an inhibitor of the PI3K pathway and (ii) hAMH or fragments thereof, wherein the subject is suffering from caner and is undergoing treatment with chemotherapy or was treated with chemotherapy.

In some embodiments, the methods and uses of the invention employ administering to a subject a combination comprising (i) an inhibitor of the PI3K pathway and (ii) a polypeptide denoted by at least one of SEQ ID NO:1, SEQ ID NO:3, SEQ ID NO:4, SEQ ID NO:5 or SEQ ID NO:6, wherein the subject is suffering from caner and is undergoing treatment with chemotherapy or was treated with chemotherapy.

In some embodiments, the methods and uses of the invention employ administering to a subject a combination comprising (i) at least one mTOR inhibitor and (ii) hAMH, or fragments, wherein the subject is suffering from caner and is undergoing treatment with chemotherapy or was treated with chemotherapy.

In some embodiments, the methods and uses of the invention employ administering to a subject a combination comprising (i) at least one mTOR inhibitor being at least one of Formulas I, II, III, IV, V, VI, VII, VIII, IX, X, XI, XII, XIII, XIV, XV, XVI, XVII or any combination thereof and (ii) hAMH, or fragments thereof, wherein the subject is suffering from caner and is undergoing treatment with chemotherapy or was treated with chemotherapy.

In some embodiments, the methods and uses of the invention employ administering to a subject a combination comprising (i) at least one mTOR inhibitor being at least one of Formulas I, II, III, or any combination thereof and (ii) hAMH, or fragments thereof, wherein the subject is suffering from caner and is undergoing treatment with chemotherapy or was treated with chemotherapy.

In some embodiments, the methods and uses of the invention employ administering to a subject a combination comprising (i) at least one mTOR inhibitor and (ii) a polypeptide denoted by at least one of SEQ ID NO:1, SEQ ID NO:3, SEQ ID NO:4, SEQ ID NO:5 or SEQ ID NO:6, wherein the subject is suffering from caner and is undergoing treatment with chemotherapy or was treated with chemotherapy.

In some embodiments, the methods and uses of the invention employ administering to a subject a combination comprising (i) at least one mTOR inhibitor being at least one of Formulas I, II, III, IV, V, VI, VII, VIII, IX, X, XI, XII, XIII, XIV, XV, XVI, XVII or any combination thereof and (ii) a polypeptide denoted by at least one of SEQ ID NO:1, SEQ ID NO:3, SEQ ID NO:4, SEQ ID NO:5 or SEQ ID NO:6, wherein the subject is suffering from caner and is undergoing treatment with chemotherapy or was treated with chemotherapy.

In some embodiments, the methods and uses of the invention employ administering to a subject a combination comprising (i) at least one mTOR inhibitor being at least one of Formulas I, II, III, or any combination thereof and (ii) a polypeptide denoted by at least one of SEQ ID NO:1, SEQ ID NO:3, SEQ ID NO:4, SEQ ID NO:5 or SEQ ID NO:6, wherein the subject is suffering from caner and is undergoing treatment with chemotherapy or was treated with chemotherapy.

In some embodiments, the female subject having cancer is being treated with anti-cancer therapy prior to and/or in parallel and/or after to the combination therapy.

In other words, in certain cases the subject to be treated with the combination of the invention is one who is treated with anti-cancer therapy. In other cases, the subject to be treated with the combination of the invention is one who was treated with anti-cancer therapy. In further cases, the subject to be treated with the combination of the invention is one who will be treated with anti-cancer therapy after being treated with the combination.

As appreciated, the amount and frequency of administration of the combination is determined in consideration of the anti-cancer therapy as well as each one of the components of the combination. In some embodiments, the anti-cancer therapy is chemotherapy.

The combination of the invention may be applicable by various dosage regimens.

The dosage regimen of the combination involves determining frequency of administration, dose per a single administration, time interval between administrations, duration of treatments, and administration route and may affect treatment outcome and specifically the inhibition of premature follicles loss and POI.

Determining the dosage regimen of the combination of the invention may depend on several factors, including, inter alia, the subject's age and overall condition or the cause of at least one of primordial follicles activation, ovarian follicles depletion, POI, destruction of the oocyte pool, follicle apoptosis, reduced infertility (increase fertility) or combination thereof.

In such embodiments, in which the IOP is due to a medical procedure, the dosing regimen may depend on the type of the procedure, for example type and severity of the procedure. As appreciated, in cases that the medical procedure is an anti-cancer treatment, the dosing regimen of the combination may depend on the administration protocol of the anti-cancer treatment.

Specifically, in certain cases the subject to be treated is one who, at the time of assessment, is treated with an anti-cancer treatment, and the amount, the frequency of administration of the combination is determined in consideration of the concurrent anti-cancer treatment. In some embodiments, the subject is one who is predisposed, suspected or known to suffer from anti-cancer side effects, whereby the administration of the combination, assists in reducing or diminishing such side effects as described herein.

In some embodiments, the at least one mTOR inhibitor and the AMH are administered separately.

In some embodiments, the at least one mTOR inhibitor is administered prior to the administration of AMH.

In some embodiments, the at least one mTOR inhibitor is administered after the administration of AMH.

In some embodiments, administration of the at least one mTOR inhibitor partially overlap with the administration of AMH. In other words, the administration of the at least one mTOR inhibitor and the administration of AMH may be at the same time.

Hence, in accordance with some aspect, the present disclosure provides a kit, the kit comprising in separate reservoirs an effective amount of at least one modulator of an intrinsic pathway as described herein and an effective amount of at least one modulator of an extrinsic pathway as described herein.

In other words, the at least one modulator of an intrinsic pathway and the at least one modulator of an extrinsic pathway may each be provided in a separate pharmaceutical dosage form.

The invention can further refer in terms of a kit comprising in separate reservoirs the at least two modulators. One reservoir comprises an effective amount of hAMH, any fragments and peptides thereof as described herein and in a different reservoir an effective amount of at least one mTOR inhibitor as described herein.

In accordance with some aspects which can be implemented as embodiments of the invention, it is provided a kit comprising (i) at least one mTOR inhibitor or a pharmaceutical composition or formulation thereof, optionally, in a first dosage form; (ii) hAMH, any fragments and peptides thereof or a pharmaceutical composition or formulation thereof optionally, in a second dosage form.

In accordance with some aspects which can be implemented as embodiments of the invention, it is provided a kit comprising: (i) a first packaging containing at least one mTOR inhibitor or a pharmaceutical composition comprising the mTOR inhibitor; (ii) hAMH, any fragments and peptides thereof or a pharmaceutical composition comprising hAMH, any fragments and peptides thereof.

In some embodiments, the kit further comprising instructions for using the effective amounts in a combination therapy.

In yet some further embodiments, the invention provides kits for use in a method for reducing, decreasing, inhibiting or preventing at least one of primordial follicles activation, ovarian follicles depletion, POI, destruction of the oocyte pool, follicle apoptosis in a subject.

In yet some further embodiments, the invention provides kits for use in a method for increasing fertility in a subject.

In some embodiments, the kit of the invention comprises at least one anti-cancer agent.

It should be noted that the kit may optionally further comprise container means for containing the compositions, formulations thereof.

According to some embodiments, the kit of the invention may further comprise container means for containing the different components of the kit of the invention or any dosage forms thereof.

The term “container” as used herein refers to any receptacle capable of holding at least one component of a pharmaceutical composition or formulation of the invention. Such a container may be any jar, vial or box known to a person skilled in the art and may be made of any material suitable for the components contained therein and additionally suitable for short or long term storage under any kind of temperature. In addition, the kit includes container means for containing separate compositions; such as a divided bottle or a divided foil packet however, the separate compositions may also be contained within a single, undivided container. Typically, the kit includes directions for the administration of the separate components, compounds or agents. As noted above, the kit form is particularly advantageous when the separate components, compounds or agents are preferably administered in different dosage forms (e.g., oral and parenteral), are administered at different dosage intervals, or when titration of the individual components of the combination is desired by the prescribing physician.

In some embodiments, the different reservoirs are different syringes or different formulation containers comprising the actives in solid or liquid or solution forms.

In some embodiments, the kit comprises instructions for combining the two pharmaceutical dosage forms (unit dose) one into the other to form a composition.

In some embodiments, the kit comprises a container and instructions to combine the two pharmaceutical dosage forms into the container.

In some embodiments, the kit comprises as single component (i.e. unit dose) comprising the at least one modulator of an intrinsic pathway and the at least one modulator of an extrinsic pathway.

In some embodiments, the kit comprises a single composition comprising the at least one modulator of an intrinsic pathway and the at least one modulator of an extrinsic pathway. In some embodiments, the kit comprises instructions as noted herein above.

In some embodiments, the at least two modulators of the combinations are administered concomitantly.

In some embodiments, the two modulators are administered in a single unit dose; namely in a unit which is suitable for administration to the subject in need thereof as detailed herein below. The unit dose can contain an effective amount, e.g. a prescribed quantity of the at least two modulators sufficient to produce a therapeutic effect.

In other words, the at least two modulators can be administered in a single composition.

Hence, some further aspect of the present disclosure relates to a pharmaceutical composition comprising as an active ingredient at least one modulator of an intrinsic pathway and the at least one modulator of an extrinsic pathway. The composition optionally further comprises at least one of pharmaceutically acceptable carrier/s, excipient/s, auxiliaries, and/or diluent/s.

In some embodiments, the pharmaceutical composition is for use in reducing, decreasing, inhibiting or preventing at least one of primordial follicles activation, ovarian follicles depletion, POI, destruction of the oocyte pool, follicle apoptosis or any combination thereof in a subject.

In some embodiments, the pharmaceutical composition is for use in increasing fertility in a subject.

The combination, kits and pharmaceutical composition of the invention can be administered and dosed by the methods of the invention, in accordance with good medical practice. More specifically, the combinations and/or compositions used in the methods and kits of the invention, described herein, may be adapted for administration by systemic, parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s).

The phrases “systemic administration,” “administered systemically,” “peripheral administration” and “administered peripherally” as used herein mean the administration of a compound, drug or other material other than directly into the central blood system, such that it enters the patient's system and, thus, is subject to metabolism and other like processes. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

Regardless of the route of administration selected, the combinations of the present invention, which may be used in a suitable hydrated form, and/or the combinations and/or pharmaceutical compositions of the present invention, are formulated into pharmaceutically-acceptable dosage forms by conventional methods known to those of skill in the art.

The forms of the combinations or pharmaceutical compositions suitable for injection use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating action of microorganisms, such as bacteria and fungi.

Sterile injectable solutions are prepared by incorporating the combinations' components in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

In the case of sterile powders for the preparation of the sterile injectable solutions, the preferred method of preparation are vacuum-drying and freeze drying techniques which yield a powder of the active ingredient plus any additional desired ingredient from a previously sterile-filtered solution thereof.

Combinations, kits and pharmaceutical compositions used according to the invention generally comprise a buffering agent, an agent who adjusts the osmolarity thereof, and optionally, one or more pharmaceutically acceptable carriers, excipients and/or additives as known in the art. Supplementary active ingredients can also be incorporated into the compositions. The carrier can be solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

Local administration to the area in need may be achieved by, for example, local infusion during surgery, topical application, direct injection into the specific organ, etc.

Compositions and combinations for oral administration include powders or granules, suspensions or solutions in water or non-aqueous media, capsules, sachets, lozenges (including liquid-filled), chews, multi- and nano-particulates, gels, solid solution, liposome, films, ovules, sprays or tablets. Thickeners, flavoring agents, diluents, emulsifiers, dispersing aids or binders may be desirable.

Pharmaceutical compositions and combinations used in subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions and combinations of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome-containing formulations.

It should be understood that in addition to the ingredients particularly mentioned above, the formulations may also include other agents conventional in the art having regard to the type of formulation in question, for example those suitable for oral administration may include flavoring agents.

In particular embodiments, the unit dosage formulations are those containing a daily dose or sub-dose, as herein above recited, or an appropriate fraction thereof, of an active ingredient.

The combinations of the invention may be administered by appropriate administration/schedule regimen.

In some embodiments, the combinations may be administrated at least twice daily, at least once daily, at least once in two days, or at least once a week.

In some embodiments, the combination or any component thereof (e.g. mTOR inhibitor and AMH) is adapted for use before, simultaneously with, after or any combination thereof with the at least one anti-cancer treatment.

In some embodiments, the methods and uses of the invention comprises sequential administering the agonist of AMHR2, the anti-cancer therapy and the at least one of mTOR inhibitor.

In some embodiments, the methods and uses of the invention comprise sequential administering said agonist of mTOR inhibitor, said anti-cancer therapy and said agonist of AMHR2.

In some embodiments, the female subject having cancer is being treated with five administration of Tem for 7 days (at day −7, −5, −3, −1 and 0), at the 7^{th} day, the subject is treated with a single dose of Cy, followed by administrations of a peptide comprising an amino acid sequence denoted by SEQ ID NO:3 every 6 hours for 24 hours.

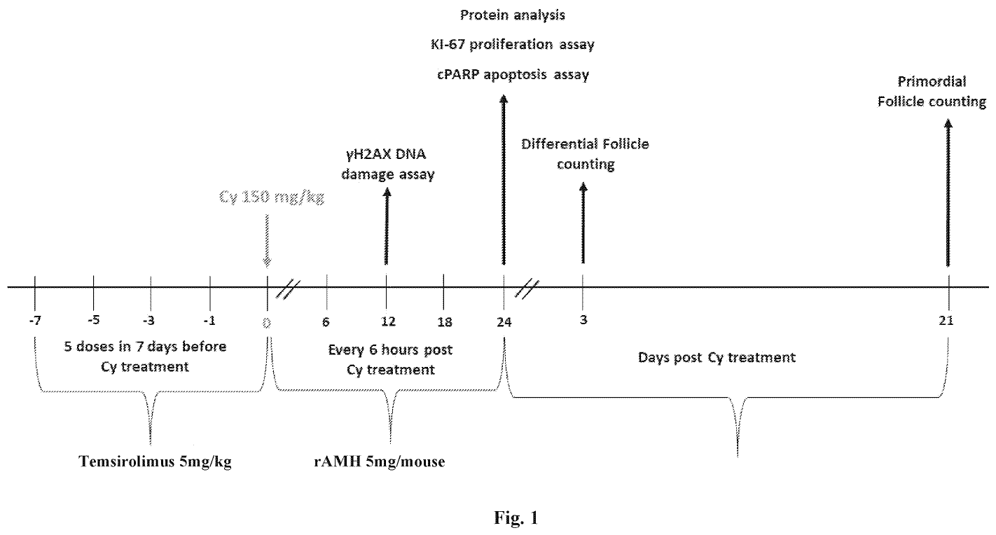

In some embodiments, the dosing regimen is as provided in FIG. 1.

In some embodiments, the female subject having cancer is being treated with anti-cancer therapy after completion of treatment with hAMH, any fragments and peptides thereof, and prior to initiation of treatment with at least one of mTOR inhibitor. In some embodiments, the anti-cancer therapy is chemotherapy.

In some embodiments, the female subject having cancer is being treated with anti-cancer therapy after completion of treatment with the combination of the invention. In some embodiments, the anti-cancer therapy is chemotherapy.

In some embodiments, the female subject having cancer is being treated with anti-cancer therapy before initiation of treatment with the combination of the invention. In some embodiments, the anti-cancer therapy is chemotherapy.

As noted herein, the combinations of the invention comprise at least two complementary modulators. The at least two complementary modulators can have a synergistic activity and hence the combination of the invention may be considered in accordance with some aspects, a synergistic combination.

In some embodiments, the methods of the invention employ administering to a subject suffering from primordial follicles activation, ovarian follicles depletion, POI, destruction of the oocyte pool, follicle apoptosis, reduced fertility or combination thereof a therapeutically effective amount of a synergistic combination comprising (i) at least one mTOR inhibitor having any one of Formulas I, II, III, IV, V, VI, VII, VIII, IX, X, XI, XII, XIII, XIV, XV, XVI, XVII or any combination thereof and (ii) a polypeptide denoted by at least one of SEQ ID NO:1, SEQ ID NO:3, SEQ ID NO:4, SEQ ID NO:5 or SEQ ID NO:6. In some embodiments, the subject is suffering from caner and undergoing treatment with chemotherapy or was treated with chemotherapy.

A synergistic combination as used herein refers to a quantity of a combination comprising at least one at least one mTOR inhibitor and hAMH as defined herein that is statistically significantly more effective than the additive effects of the at least one mTOR inhibitor and hAMH when used individually (e.g. not in combination). A “synergistic amount” for a component in a combination (e.g. at least one mTOR inhibitor and hAMH) is defined as an amount providing a synergistic effect.

A determination of a synergistic interaction between the at least one mTOR inhibitor and hAMH may be based on the results obtained from the assays known in the art.

For example, synergism can be quantitated by calculating a synergy factor (SF) which is defined as SF=OR₁₂/(OR₁×OR₂), wherein OR₁ is the effect of the mTOR inhibitor alone, OR₂ is the effect of the hAMH and OR₁₂ is the effect of both (i.e. the combination). Synergism is defined for SF>1.

As noted herein, the combination comprising an mTOR inhibitor that is a small molecule. A small molecule as used herein may encompass at least one of solvate, a hydrate, a stereoisomer, a pharmaceutically acceptable prodrug, a pharmaceutically active metabolite, a pharmaceutically acceptable salt, a crystalline form, an amorphous form, a physiologically functional derivative.

The term “solvate” refers to an aggregate of a molecule with one or more solvent molecules, such as hydrate, alcoholate (aggregate or adduct with alcohol), and the like.

The term “hydrate” refers to a compound formed by the addition of water. The hydrates may be obtained by any known method in the art by dissolving the compounds in water and recrystallizing them to incorporate water into the crystalline structure.

The term “stereoisomer” as used herein encompasses an “enantiomer”, the enantiomer refers to a compound that is superposable with respect to its counterpart only by a complete inversion/reflection (mirror image) of each other. The small molecule in accordance with the present disclosure encompass any enantiomers (i.e. R or S).

The term “prodrug” or “pharmaceutically acceptable prodrug” as used herein refers to a compound that may be converted under physiological conditions to the specified compound or to a pharmaceutically acceptable salt of such compound. Prodrugs may be useful for facilitating the administration of a parent drug.

The term “metabolite” or “pharmaceutically acceptable metabolite” as used herein refers to a compound that is formed under physiological conditions to of degrading and eliminating the compounds. Oxidative metabolite may an example.

The term “pharmaceutically acceptable salt” refers to salts derived from organic and inorganic acids of a compound described herein. Exemplary salts include, but are not limited to, sulfate, citrate, acetate, oxalate, chloride, hydrochloride, bromide, hydrobromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, succinate, fumarate, maleate, malonate, mandelate, malate, phthalate, and pamoate. The term “pharmaceutically acceptable salt” as used herein also refers to a salt of a compound described herein having an acidic functional group, such as a carboxylic acid functional group, and a base. Exemplary bases include, but are not limited to, hydroxide of alkali metals including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like. The term “pharmaceutically acceptable salt” also includes hydrates of a salt of a compound described herein.

A crystalline and/or amorphous forms of the small compounds described herein include, for example, polymorphs, pseudopolymorphs, solvates, hydrates, unsolvated polymorphs (including anhydrates), conformational polymorphs, and amorphous forms of the compounds, as well as mixtures thereof.

The term “physiologically functional derivative” used herein relates to any physiologically acceptable derivative of a compound as described herein. The physiologically functional derivatives also include prodrugs of the compounds of the invention. As noted herein, such prodrugs may be metabolized in vivo to a compound of the invention. These pro-drugs may or may not be active themselves and are also an object of the present invention.

The term “derivative” in accordance with the small molecule of the present invention also encompasses chemically modified small molecule derived from a parent compound of the invention that differs from the parent compound by one or more elements, substituents and/or functional groups such that the derivative has the same or similar biological properties/activities as the parent compound.

As noted herein, the present disclosure encompasses AMH, a derivative, an analogue, a variant or a homologue thereof as well as a derivative of AMH analogue, AMH variant or AMH homologue, specifically a derivative of hAMH analogue, hAMH variant or hAMH homologue.

The term “derivative” in connection with a polypeptide chain is used to define amino acid sequences (polypeptide), with any insertions, deletions, substitutions and modifications to the amino acid sequences (polypeptide) that do not alter the activity of the original polypeptides. By the term “derivative” it is also referred to homologues, variants and analogues thereof, as well as covalent modifications of a polypeptides made according to the present invention.

In some embodiments, derivatives include, but are not limited to, polypeptides that differ in one or more amino acids in their overall sequence from the polypeptides defined herein (either the AMH protein or any fragment or peptide derived therefrom according to the invention), polypeptides that have deletions, substitutions, inversions or additions.

In some embodiments, derivatives refer to polypeptides, which differ from the polypeptides specifically defined in the present invention by insertions of amino acid residues. It should be appreciated that by the terms “insertions” or “deletions”, as used herein it is meant any addition or deletion, respectively, of amino acid residues to the polypeptides used by the invention, of between 1 to 50 amino acid residues, between 20 to 1 amino acid residues, and specifically, between 1 to 10 amino acid residues. More particularly, insertions or deletions may be of any one of 1, 2, 3, 4, 5, 6, 7, 8, 9, or 10 amino acids. It should be noted that the insertions or deletions encompassed by the invention may occure in any position of the modified peptide, as well as in any of the N-terminal or C-terminal thereof.

The peptides that may be used by the methods of the invention may all be positively charged, negatively charged or neutral. In addition, they may be in the form of a dimer, a multimer or in a constrained conformation, which can be attained by internal bridges, short-range cyclizations, extension or other chemical modifications.

The polypeptides used by the methods of the invention can be coupled (conjugated) through any of their residues to another peptide or agent. For example, the polypeptides of the invention can be coupled through their N-terminus to a lauryl-cysteine (LC) residue and/or through their C-terminus to a cysteine (C) residue.

Further, the peptides may be extended at the N-terminus and/or C-terminus thereof with various identical or different amino acid residues. As an example for such extension, the peptide may be extended at the N-terminus and/or C-terminus thereof with identical or different amino acid residue/s, which may be naturally occurring or synthetic amino acid residue/s. An additional example for such an extension may be provided by peptides extended both at the N-terminus and/or C-terminus thereof with a cysteine residue. Naturally, such an extension may lead to a constrained conformation due to Cys-Cys cyclization resulting from the formation of a disulfide bond. Another example may be the incorporation of an N-terminal lysyl-palmitoyl tail, the lysine serving as linker and the palmitic acid as a hydrophobic anchor.

In addition the peptides may be extended by aromatic amino acid residue/s, which may be naturally occurring or synthetic amino acid residue/s, for example, a specific aromatic amino acid residue may be tryptophan. The peptides may be extended at the N-terminus and/or C-terminus thereof with various identical or different organic moieties, which are not naturally occurring or synthetic amino acids. As an example for such extension, the peptide may be extended at the N-terminus and/or C-terminus thereof with an N-acetyl group.

For every single peptide sequence defined by the invention and disclosed herein, this invention includes the corresponding retro-inverse sequence wherein the direction of the peptide chain has been inverted and wherein all the amino acids belong to the D-series.

The invention also encompasses any homologues of the polypeptides (either the AMH protein or any fragments or peptides thereof) specifically defined by their amino acid sequence according to the invention. The term “homologues” is used to define amino acid sequences (polypeptide) which maintain a minimal homology to the amino acid sequences defined by the invention, as further defined herein above.

“Homology” with respect to a native polypeptide and its functional derivative is defined herein as the percentage of amino acid residues in the candidate sequence that are identical with the residues of a corresponding native polypeptide, after aligning the sequences and introducing gaps, if necessary, to achieve the maximum percent homology, and not considering any conservative substitutions as part of the sequence identity. Neither N- nor C-terminal extensions nor insertions or deletions shall be construed as reducing identity or homology. Methods and computer programs for the alignment are well known in the art.

In some embodiments, the present invention also encompasses polypeptides which are variants of, or analogues to, the polypeptides specifically defined in the invention by their amino acid sequence. With respect to amino acid sequences, one of skill will recognize that individual substitutions, deletions or additions to peptide, polypeptide, or protein sequence thereby altering, adding or deleting a single amino acid or a small percentage of amino acids in the encoded sequence is a “conservatively modified variant”, where the alteration results in the substitution of an amino acid with a chemically similar amino acid.

Conservative substitution tables providing functionally similar amino acids are well known in the art. Such conservatively modified variants are in addition to and do not exclude polymorphic variants, interspecies homologues, and alleles and analogous peptides of the invention.

For example, substitutions may be made wherein an aliphatic amino acid (G, A, I, L, or V) is substituted with another member of the group, or substitution such as the substitution of one polar residue for another, such as arginine for lysine, glutamic for aspartic acid, or glutamine for asparagine. Each of the following eight groups contains other exemplary amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)

More specifically, amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar “hydrophobic” amino acids are selected from the group consisting of Valine (V), Isoleucine (I), Leucine (L), Methionine (M), Phenylalanine (F), Tryptophan (W), Cysteine (C), Alanine (A), Tyrosine (Y), Histidine (H), Threonine (T), Serine (S), Proline (P), Glycine (G), Arginine (R) and Lysine (K); “polar” amino acids are selected from the group consisting of Arginine (R), Lysine (K), Aspartic acid (D), Glutamic acid (E), Asparagine (N), Glutamine (Q); “positively charged” amino acids are selected form the group consisting of Arginine (R), Lysine (K) and Histidine (H) and wherein “acidic” amino acids are selected from the group consisting of Aspartic acid (D), Asparagine (N), Glutamic acid (E) and Glutamine (Q).

Each component in the combination of the invention is administered in an effective amount such that the combination is effective as described herein. The term “effective amount” relates to the amount of an active agent, that is needed to provide a desired level of active agent in the bloodstream or at the site of action in an individual to be treated to give an anticipated physiological response when such composition is administered. The precise amount will depend upon numerous factors, e.g., the active agent, the activity of the composition, the delivery device employed, the physical characteristics of the composition, intended patient use (i.e., the number of doses administered per day), patient considerations, and the like, and can readily be determined by one skilled in the art, based upon the information provided herein. As used herein, an “effective amount” of the combination encompasses an effective amount of each one of the components of the combination, i.e. a modulator of an intrinsic pathway and a modulator of an extrinsic pathway, and is meant any amount effective for the reducing, decreasing, inhibiting or preventing at least one of primordial follicles activation, ovarian follicles depletion, POI, destruction of the oocyte pool, follicle apoptosis or any combination thereof as well as for increasing fertility as disclosed herein.

The terms “inhibition”, “moderation”, “reduction” or “attenuation” as referred to herein, relate to reduction of at least one of primordial follicles activation, ovarian follicles depletion, premature ovarian insufficiency (POI), destruction of the oocyte pool, follicle apoptosis by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9% as compared to a subject not treated with the combination of the invention or control subject.

The terms “increase”, “elevation”, “enhancement” or “elevation” as referred to herein, relate to the enhancement and increase of the PMF reserve and/or fertility of a subject, specifically, a mammalian subject, by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, as compared to a subject not treated with the combination of the invention or control subject.

With regards to the above, it is to be understood that, where provided, percentage values such as, for example, 10%, 50%, 120%, 500%, etc., are interchangeable with “fold change” values, i.e., 0.1, 0.5, 1.2, 5, 10, 20, 30, 40, 50, 60, 70, 80, 90, 100 etc., respectively.

As used herein, the forms “a”, “an” and “the” include singular as well as plural references unless the context clearly dictates otherwise. For example, the term “an antagoist” includes one or more modulators.

Further, as used herein, the term “comprising” is intended to mean that the composition include the recited components, e.g. at least one modulator. “Consisting of” shall thus mean excluding more than trace amounts of other components. Embodiments defined by each of these transition terms are within the scope of this invention.

Further, all numerical values, e.g. when referring the amounts or ranges of the components, are approximations which are varied (+) or (−) by up to 20%, at times by up to 10%, at times up to 5% of from the stated values. It is to be understood, even if not always explicitly stated that all numerical designations are preceded by the term “about”.

It should be noted that various embodiments of this invention may be presented in a range format. The description of a range should be considered to have specifically disclosed all the possible sub ranges as well as individual numerical values within that range. For example, description of a range such as from 1 to 6 or between 1 and 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6.

It should be further noted that the various embodiments and examples detailed herein in connection with various aspects of the invention may be applicable to one or more aspects disclosed herein. It should be further noted that any embodiment described herein, for example, related to combinations of the invention, may be applied separately or in various combinations as well as may be applied separately or in various combinations to the uses, compositions, kits and methods. Various embodiments and aspects of the present invention as delineated hereinabove and as claimed in the claims section below find experimental support in the following examples. The phrases “in another embodiment” or any reference made to embodiment as used herein do not necessarily refer to different embodiment, although it may. Thus, various embodiments of the invention can be combined (from the same or from different aspects) without departing from the scope of the invention.

The invention will now be exemplified in the following description of experiments that were carried out in accordance with the invention. It is to be understood that these examples are intended to be in the nature of illustration rather than of limitation. Obviously, many modifications and variations of these examples are possible in light of the above teaching. It is therefore, to be understood that within the scope of the appended claims, the invention may be practiced otherwise, in a myriad of possible ways, than as specifically described hereinbelow.

### NON-LIMITING EXAMPLES

### Materials and Methods

### In Vivo Experiments:

BALB/c female mice aged 12 days or 8 weeks (Envigo, Israel) were given a single intraperitoneal injection of 200 μl of PBS or an equal volume of 150 mg/kg Cy (Sigma-Aldrich), 5 mg/kg temsirolimus (Selleck Chemicals; 5 doses in 7 days before Cy treatment, Bozec et al., 2011; Wan et al., 2006) or 5 μg/mouse human C-terminus recombinant AMH (rAMH R&D Systems), 5 doses in 24 hours—every 6 hours following the administration of Cy (Roness et al., 2019). Ovaries were collected either 12 or 24 hours after Cy treatment for protein analysis and histochemical staining, and 3 or 21 days after Cy treatment for H&E staining and follicle counts. Ovaries were removed and placed immediately into either RNA later solution or 4% paraformaldehyde. All procedures were approved by the Institutional Animal Care and Use Institutional Ethics Committee.

### Ex Vivo Experiments:

Female 12 days BALB/c mice were sacrificed, and their ovaries were removed and transferred to Transwell culture dishes (6 well plates, 16-18 ovaries per well) with media alone (DMEM F12 media containing 1 mg/ml BSA, 1 mg/ml Albumax, 50 μg/ml Ascorbic acid, 10 ng/ml Insulin, 0.1 mg/ml L-Glutamine, 0.1 mg/ml pen-strep, and 2.5 μg/ml transferrin) or media with 400 nM Tem. Plates containing ovaries were cultured at 37° C. in humidified air with 5% CO₂. After 2 hours in culture, mice were randomly allocated to one of 4 treatment groups: 1. Ovaries in media alone 2. Cy metabolite, Phosphoramide Mustard CS (PM, 20 μM) was added to ovaries in media for 4 hours, then PM was removed, and ovaries were recultured in media alone. 3. Ovaries in media plus Tem alone. 4. PM was added to ovaries in media plus Tem for 4 hours, then PM was removed, and ovaries were recultured in media plus Tem. Seven days after PM was added, 6 ovaries from each group were fixed in Paraformaldehyde (PFA) for follicle counting (H&E). Media of all groups were changed daily.

### Histology

Ovaries removed 12 h, 24h, 3 days or 21 days following Cy treatment were fixed in PFA, paraffin-embedded and serially sectioned (5-μm).

Follicle counting: H&E staining was used for differential follicle counts in ovaries removed 3 and 21 days following Cy treatment and conducted as previously described. (Briefly, blind follicle counts were conducted on every tenth section of entire ovaries. Follicle stage was classified according to accepted definitions: a PMF was counted when the nucleus was identified surrounded by a single layer of flattened squamous pre-granulosa cells, a primary follicle was defined as an oocyte surrounded by a single layer of cuboidal granulosa cells, a secondary follicle had two or more layers of cuboidal granulosa cells, but no antrum, antral follicles had an antrum and were counted only if the oocyte was visible to avoid double counting. Follicles were classified as: primordial, primary or secondary plus (i.e., secondary and antral) follicles, and these were also grouped into dormant (primordial) or growing (primary, secondary and antral) follicles. Atretic follicles were not calculated. Only follicles with non-atretic and healthy granulosa cells were counted for total number

Proliferation assay: Sample sections (5μ) from ovaries removed 24h after Cy treatment were stained for proliferation with Ki-67 antibody (Cat No: 275R-14, Cell marque). At this time interval the effect of treatment is mainly observed on primary follicles, which were counted and categorized as follows: No proliferation—no Ki-67 staining in granulosa cells. Mild proliferation: 1-2 granulosa cells per follicle stained for Ki-67. Extensive proliferation: 3 or more granulosa cells per follicle stained for Ki-67. An average number of primary follicles stained in each category in 2-3 sections per ovary was calculated.

DNA damage assay: Sample sections (5μ) from ovaries removed 12h after Cy treatment were stained for DNA damage evaluation with γH2AX antibody, and all the PMF oocytes were classified as positive/negative for DNA damage. Oocytes were counted from 6-8 ovaries and the ratio of stained oocytes to total oocytes was calculated.

Apoptosis assay: Sample sections (5μ) from ovaries removed 24h after Cy treatment were stained for apoptotic marker cPARP antibody, and all the PMF oocytes were classified as positive/negative for apoptosis. Oocytes were counted from 6-8 ovaries and the ratio of stained oocytes to total oocytes was calculated.

### Western Blotting

For protein analysis, ovaries were snap frozen in liquid nitrogen and stored at −80°. Ovaries were homogenized with 60 μl RIPA buffer with Protease/Phosphatase Inhibitors and centrifuged at 14,000 rpm in 40 for 20 minutes. Protein concentration was determined using a bicinchoninic acid assay, and 40 μg of protein was loaded onto 10% SDS polyacrylamide gels. After transferring proteins to nitrocellulose membranes, blots were probed with appropriate antibodies—Akt (C67E7) [rabbit monoclonal antibody (mAb) Cat #4691], phospho-Akt (Ser473) (D9E) (XP rabbit mAbCat #4060), rpS6 (5G10) (rabbit mAb 2217), phospho-rpS6 (Ser235/236) (antibody 2211), mTOR (7C10) (rabbit mAb 2983), phospho-mTOR (Ser2448) (antibody 2971), phospho-FOXO3a (Thr32) (antibody 9464) (all from Cell Signaling Technology), vinculin or anti-mouse β-Actin (Sigma-Aldrich). Integrated light intensity of each band was quantified with Image Lab 6.0.1 software and used to compare treatment-induced changes in the concentration of phosphoproteins and to calculate the ratio of phosphorylated proteins to their non-phosphorylated forms/housekeeping proteins. Vinculin or β-Actin were used to normalize the intensity values of the phosphoproteins.

### Statistical Analysis

A mixed model for repeated measurements (Verbeke and Molenberghs, 2000) was used to compare the different treatment groups. Comparisons were calculated using the mixed procedure in SAS (version 9.1.3; SAS Institute, Inc., Cary, NC) and were adjusted for multiple comparisons using the Tukey-Kramer test. P<0.05 was considered to be significant.

### Results

### Example 1: Tem or rAMH Alone Attenuate Follicle Loss in Ex Vivo Culture and In Vivo Mouse Models of Chemotherapy

For initial testing of the functional ability of Tem or rAMH to prevent Cy-induced follicle loss, the inventors used an ex vivo model of chemotherapy-induced follicle activation, culturing neonatal mouse ovaries in the presence of PM, with or without the addition of Tem or rAMH.

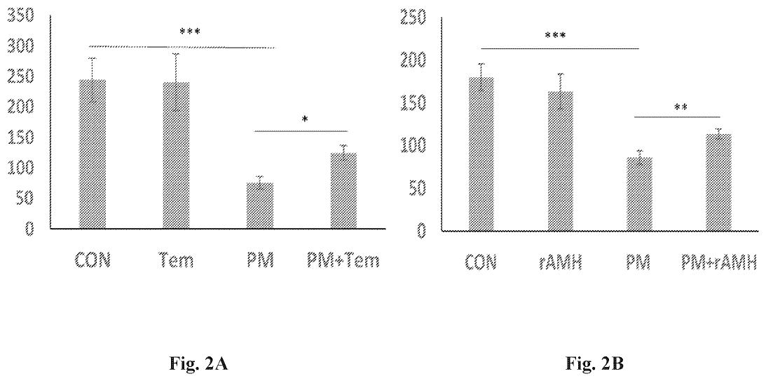

Ovaries cultured with PM alone had significantly fewer PMFs on day 7 of culture compared to those cultured in control media; 75±5.1 compared to 244±17.8 (FIG. 2A, p<0.001). This follicle loss was attenuated in ovaries co-cultured with Tem or with rAMH. Ovaries co-cultured with Tem had significantly more PMFs on day 7 of culture compared to PM alone; 124±5.9 vs 75±5.1 (FIG. 2A, p<0.05), as did those co-cultured with rAMH, (113±4.6 compared to 86±8.4 (FIG. 2B p<0.01).

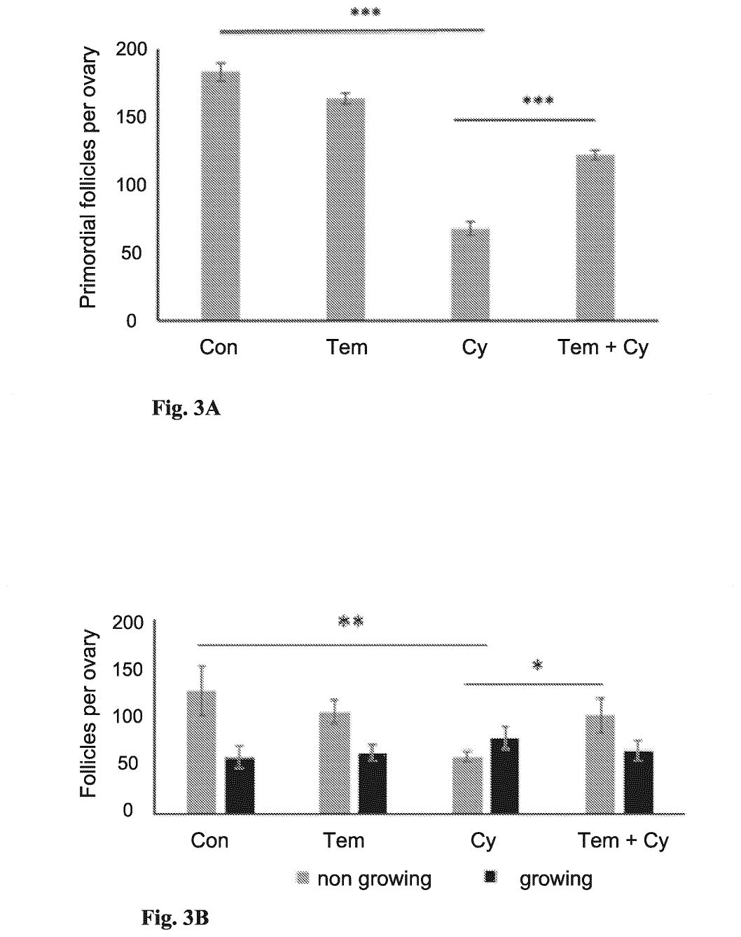

The inventors then examined the effect of co-treatment with Tem on PMF depletion induced by Cy in an in vivo mouse model. Ovaries were removed 3 days after treatment with Cy, with or without co-treatment with Tern, to examine short term changes in follicle dynamics and 21 days following exposure to examine long term impact on the follicle reserve. Total follicle counts at 21 days showed that 150 mg/kg Cy caused significant reduction in PMF reserve (67.8*2.5 compared to 183±3.32 in untreated ovaries [FIG. 3A p<0.001]). Ovaries from mice which received co-treatment with Tem retained a significantly higher number of PMFs compared to those treated with Cy alone, 122.5±1.7, (FIG. 3A, p<0.001). This was similar to previous data from the inventor's lab which showed that in vivo co-treatment with rAMH conveyed a significant measure of protection to the ovarian follicle reserve, which retained 34% more PMFs 21 days post treatment compared to ovaries treated with Cy alone (Roness, 2019).

Follicle counts in ovaries removed 3 days after Cy treatment also showed a decrease in PMFs in Cy group compared to control and Tem co-treated ovaries, although to a lesser degree: 59.5±2.7 vs 127.2±12.5 and 101.6±8.9 respectively, (FIG. 3B, p<0.01 and p<0.05, respectively). At this time point, there was an increase in the total number of growing follicles in ovaries treated with Cy alone; 78.5±6.1 compared to 58.8±5.7 in untreated ovaries (not significant). This increase in growing follicles was not seen in ovaries co-treated with Tem, (65.6±5.2 FIG. 3B). This was reflected in the ratio between growing and dormant follicles, which was highest in Cy treated ovaries (1.31, FIG. 3C), and much lower in ovaries co-treated with Tem (0.66) and untreated ovaries (0.46, FIG. 3C).

### Example 2: Combined Co-Treatment with Tem and rAMH Provides 100% Protection Against Cy Induced Ovarian Reserve Depletion In Vivo

Using the same in vivo mouse model of chemotherapy-induced ovotoxicity, the inventors then examined whether a combination of Tem and rAMH would increase the protection of the ovarian reserve from Cy induced follicle depletion.

Three weeks after treatment, mice that received the combined treatment of Tem and rAMH with Cy had complete retention of the PMF reserve, to the extent that the number of PMFs in this group was almost identical to untreated animals (144±2.8 in control vs. 150.5±4.9 in ovaries treated with Cy+Tem+rAMH, p<0.001, FIG. 4). Co-treatment with Tern or AMH independently partially decreased the extent of follicle loss to a similar degree; PMF loss of 52%, 68.5±2.69, and 67±2.43 PMFs respectively compared to PMF loss of 77%, 32±2.12 in Cy treated ovaries (p<0.001, FIG. 4).

### Example 3: Tem Inhibits Primary Follicle Proliferation

Assessment of primary follicles proliferation using Ki-67 staining was done according to the criteria shown in FIGS. 5A-5C. Specifically, FIG. 5A no proliferation —no staining in any granulosa cells positive for Ki-67; FIG. 5B mild proliferation, 1-2 granulosa cells stained positive for Ki-67 (shown by arrows); FIG. 5C extensive proliferation, more than 3 granulosa cell stained positive for Ki-67 (shown by arrows).

FIG. 5D-5K are images showing inhibition of primary follicle proliferation by Tem, rAMH and their combination after a single dose of Cy (150 mg/kg) or the equivalent volume of PBS, FIGS. 5D and 5E ovaries administered with PBS without and with administration of Cy, respectively; FIGS. 5F and 5G ovaries administered with Tem without and with administration of Cy, respectively; FIGS. 5H and 5I ovaries administered with rAMH without and with administration of Cy, respectively; FIGS. 5 and 5K ovaries administered with Tem and rAMH without and with administration of Cy, respectively; an analysis of ovaries from 8-week-old mice that were removed 24 hours after treatment and sample sections were used for immunohistochemistry and stained for proliferation marker Ki-67.

Summary of proliferation using the criteria shown in FIGS. 5A-5C is shown in.

TABLE 1
Granulosa cells in primary follicles (%)

| | No | Mild | Extensive |
|---|---|---|---|
| Treatment | proliferation | proliferation | proliferation |
| Control | 54 | 25 | 21 |
| Tem | 73 | 22 | 5 |
| rAMH | 45 | 37 | 18 |
| Tem + rAMH | 74 | 22 | 4 |
| Cy | 16 | 63 | 21 |
| Cy + Tem | 68 | 24 | 8 |
| Cy + rAMH | 43 | 38 | 19 |
| Cy + Tem + rAMH | 65 | 26 | 9 |

As shown in Table 1, ovaries treated with Cy alone, 84% of the primary follicles were classified as proliferating (either mild or extensive proliferation) (p<0.001,). Tem reduced the level of proliferation seen in the primary follicles, such that only 32% of primary follicles showed proliferation in ovaries co-treated with Tem (p<0.001). In particular, treatment with Tem reduced the number of primary follicles with extensive proliferation compared to Cy treated ovaries (21% vs 8%). rAMH, however, reduced primary follicle proliferation to a lesser degree than Tem. In ovaries treated with rAMH (with or without Cy) approximately 55% of primary follicles were classified as proliferating (not significant compared to control). Proliferation rate of primary follicles in ovaries treated with Cy+Tem+rAMH was similar to that of Cy+Tem.

### Example 4: Combined Treatment of Tem and rAMH Reduced Cy Induced PI3K Pathway Activation

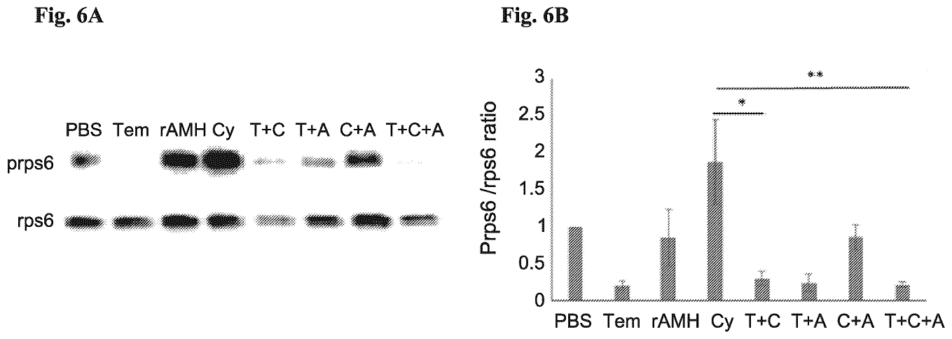

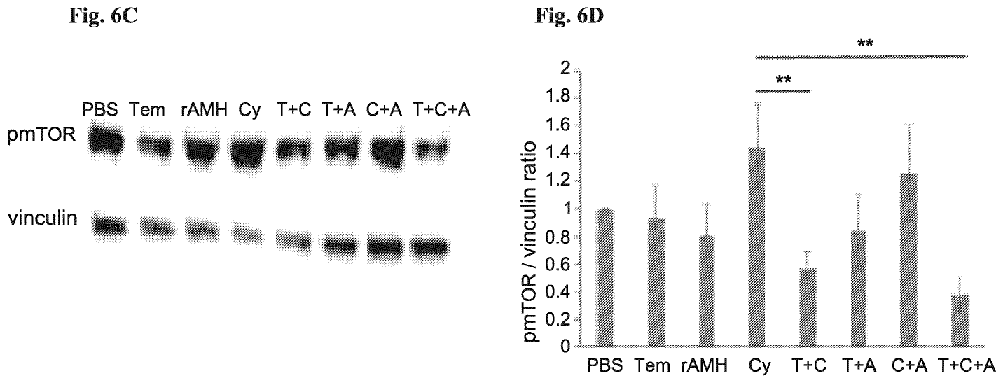

Protein analysis of whole ovaries removed 24 hours after treatment with Cy showed decreased phosphorylation of key PI3K pathway activation proteins mTOR and rpS6 when the ovaries were co-treated with Tem and rAMH. A decrease in phosphorylated form of rpS6 and mTOR was seen in ovaries exposed to Cy and Tem alone, and even greater reduction in the ovaries co-treated with Tem+rAMH (relative band intensity of 0.3 and 0.21 vs 1.88 p<0.0, p<0.01 and 0.57 and 0.38 vs 1.44, p<0.01, p<0.01 respectively, FIGS. 6A and 6B).

### Example 5: Tem and rAMH Decreased Cy-Induced DNA Damage and Direct Oocyte Death

In addition to examining the ovaries for evidence of follicle activation, the inventors also checked if there was evidence of increased apoptosis. The inventors conducted staining of the ovaries for DNA damage marker γH2AX and for apoptosis marker cPARP.

The inventors found high levels of basal DNA damage in untreated ovaries: 59% of PMF oocytes were stained positive for γH2AX (FIG. 7A). However, following Cy treatment, significant increase in DNA damage was demonstrated, when more than 90% of the PMF oocytes were stained for γH2AX (FIG. 7A). This increase in DNA damage in PMF oocytes was significantly attenuated in ovaries which received the combined co-treatment. In this treatment group only 69% of the PMF oocytes were positive for DNA damage. This was not significantly higher than in untreated ovaries.

However, despite the high levels of DNA damage evident in many of the PMF oocytes, including in untreated ovaries (59%), the inventors found that the actual levels of apoptosis in the PMF oocytes, as indicated by positive staining for cPARP, were far lower. In untreated ovaries none of the PMF oocytes stained positive for cPARP (FIG. 7B). In ovaries treated with Cy 22% of the PMF oocytes stained positive for cPARP, and this was significantly reduced in ovaries which were cotreated with Tem, or with the combined co-treatment of Tem and rAMH (14% and 12% cPARP stained PMF oocytes, respectively).

The high rates of DNA damage seen in the PMF oocytes in all treatment groups raised a concern, since the vast majority of these PMFs survived and were present 21 days after treatment. In order to assess if there was sustained DNA damage in the surviving PMF oocytes, the inventors conducted 3 cycles of mating with proven fertile males, litter sizes were evaluated, and pups were examined for physical abnormalities or malformations. The mean litter size of the mice in the co-treatment group was similar to both the untreated mice and the Cy treated mice (5.71 [n=14] vs 4.56 [n=21] and 4.62 [n=19] respectively, not significant). Pups from all the treatment groups were assessed as healthy with no gross malformations or abnormalities.

### Summary of Findings

The results of this study demonstrate that combined administration of two PMF activation pathway regulators: Temsirolimus—an intra follicular PI3K pathway inhibitor, together with rAMH—an extra follicular suppressive agent, completely prevents Cy induced follicle depletion and protects ovarian reserve in mice.

Cy has both short-term temporary and long-term persistent effects on the ovary. Direct short-term Cy effects cause apoptosis in proliferating granulosa cells of growing follicles, resulting in loss of growing follicles of all stages presented as temporary amenorrhea (Anderson and Spears 2015) and acute disruption of AMH secretion (Kano et al., 2017, Sonigo et al., 2018; Roness et al., 2019). Long-term Cy effects are secondary to loss of the dormant PMF reservoir. Significant PMF depletion after chemotherapy has been demonstrated in human ovaries, (Bath et al., 2003; Shai et al., 2021) mice (Meirow et al., 1999) and rhesus monkeys ovaries. While complete loss of PMF population results in immediate amenorrhea and permanent sterilization, partial loss will lead to gradual POI when the reduced follicle reserve is exhausted (Petrek et al., 2006; Donnez et al., 2006; Sklar et al., 2006; Partridge et al., 2010; Gracia et al., 2012). It is therefore crucial to understand the mechanisms of chemotherapy-induced loss of the PMF population.

Tem, a rapamycin macrocyclic antibiotic analog, decreases mTOR activity by inhibition of mTOR-responsive gene translation resulting in reduced proliferation (Mita et al., 2016). Studies have demonstrated that co-administration of rapamycin or its derivatives during chemotherapy partially protects ovarian reserve from Cy or cisplatin-induced PMF loss (Goldman et al., 2017; Tanaka et al., 2018; Sato and Kawamura 2020). However, an important limitation of PI3K pathway inhibition compounds (through mTOR inhibition) is their broad systemic regulatory activity in all cells (Manning and Cantley 2007). On the other hand, AMH activity in the adult female is almost entirely targeted to the ovary (Bedenk et al., 2020). Produced by granulosa cells of early growing follicles, AMH inhibits initiation of PMFs growth in rodents (Durlinger et al., 2002; Gigli et al., 2005; Yang et al., 2017) and in human ovaries, as was shown in xenotransplantation and in most in vitro studies (Carlsson et al., 2006). A single study on in vitro cultured human ovarian sections did show activation following exposure to AMH (Schmidt et al., 2005), although there has been no further evidence that this occurs. In vivo mouse studies have demonstrated that co administration of AMH during chemotherapy attenuates ovarian reserve depletion and improves fertility in mouse models (Kano et al., 2017; Sonigo et al., 2018; Roness et al., 2019). Little is known about the downstream pathways via which AMH acts on the PMFs, and it is possible that AMH suppression of follicle activation is also mediated by downregulation of the PI3K pathway and inhibition of FOXO3a phosphorylation (Sonigo et al., 2018), thereby maintaining the PMFs in a quiescent state.

This study confirms the ability of follicle activation inhibitors Tem or rAMH alone to partially attenuate chemotherapy-induced PMFs activation in both ex-vivo and in-vivo models. Each inhibitor alone protects against Cy-induced PMF stockpile depletion by 40-50%. But combined suppression of follicle activation via inhibition of both intrinsic and extrinsic pathways provided complete protection of the PMF population from Cy-induced follicle loss, resulting in ovarian morphology similar to control ovaries (FIG. 5). The suppression of pathological follicle activation was confirmed by changes seen in molecular regulators of follicle activation as well as reduction in Ki-67 immunostaining in early growing follicles in the short term following treatment. A possible connection between the two pathways might be through PI3K pathway regulator, FOXO3a. The PI3K signaling network promotes cell survival by phosphorylating FOXO3a, leading to its export from the nucleus into the cytoplasm, thus inhibiting FOXO pro-apoptotic transcription factors and promoting cellular proliferation (Jang et al., 2016). Phosphorylation of FOXO3A has also been shown to be disrupted with AMH treatment (Reddy et al., 2010). While both inhibitors reduced the PI3K pathway markers of activation and proliferation, Tem had a far stronger effect than rAMH. The fact that both inhibitors had similar degrees of protection on PMF numbers, and full protection when combined, suggests that rAMH may act primarily via an alternative PMF suppression pathway to the PI3K pathway, and implies a complementary effect between the two inhibitors.

According to the studies which propose that PMF loss is primarily attributable to direct PMF oocyte apoptotic death (Gonfloni et al., 2009; Klinger et al., 2015; Nguyen et al., 2019; Bellusci et al., 2019), chemotherapy induces double-stranded DNA breaks in the oocyte, and if not repaired, the PMF oocyte undergo apoptosis and follicular atresia within 24h of chemotherapy. Although other studies did not find evidence of apoptosis within this timeframe (Kalich-Philosoph et al., 2013; Chang et al., 2015; Jang et al., 2016; Kano et al., 2017; Zhou et al., 2017; Shai et al., 2021), our results do show evidence of significant genetic damage in almost all PMF oocytes 12h following exposure to Cy (as shown by DNA damage marker γH₂AX). However, only 22% of these subsequently underwent apoptosis (as indicated by apoptotic marker cPARP) 24h following 150 mg/kg Cy exposure. These results support a lesser role for direct apoptosis in PMF loss, but also indicate that it is not possible to equate DNA damage with oocyte death. Although most often researched separately, involvement of both follicle activation and apoptosis in PMF depletion caused by chemotherapy is plausible and was previously proposed (Bellusci et al., 2019). The data presented here indicates that less than a third of the total PMF loss can be attributed to apoptosis, implying that most of the PMF depletion is due to PMF activation but that both mechanisms play a significant role. The combined treatment of Tem and rAMH acted not just on the follicle activation pathways, but also significantly decreased DNA damage and oocyte death which may explain the maximal protective effect of the combined treatment. Links between activation of the PI3K pathway and DNA damage/apoptosis pathways have previously been shown; γH₂AX was elevated when the AKT-FOXO3 pathway was activated, and high intracellular levels of Akt have been reported to increase DNA damage, repress nuclear translocation of BRCA1 and compromise homologous recombination in breast cancer cells (Bellusci et al., 2019; Maidarti et al., 2020). Although most follicles which demonstrated DNA damage following Cy survived, there was no persistent major damage to DNA integrity or reproductive outcome, as we observed no increase in fetal resorptions or malformation rates in pregnancies following treatment with Cy or any combination of the protective agents in 3 consecutive mating rounds.

A limitation, as well as a source of conflicting evidence, in studies which investigate the mechanisms of follicle loss post-chemotherapy, is the varying timeframes which are chosen for analysis. Studies which did not show evidence of over activation (Eldani et al., 2020), may not have noticed it due to shorter time intervals between Cy administration and evaluation (12h instead of 24h or a few days), or because they used non-specific proteins such as pAkt as the marker of activation instead of more specific activation markers such as pFOXO3A, pMTOR and prpS6 (Eldani et al., 2020). In this study the inventors chose 24h following Cy administrations as the time point for exploring activation parameters and apoptosis. This time point was chosen based on other studies (Zhou et al., 2017; Goldman et al., 2017; Sonigo et al., 2018; Nguyen et al., 2019).

It is possible that the peak effect of rAMH on proliferation occurs at a later time point.

1-51. (canceled)

52. A combination comprising at least two modulators, wherein at least one of the at least two modulators inhibit the phosphatidylinositol 3-kinase (PI3K) pathway and at least one other of the at least two modulators is an agonist of anti-Mullerian hormone receptor type 2 (AMHR2).

53. The combination of claim 52, wherein said modulator that inhibits the PI3K pathway is at least one mTOR inhibitor.

54. The combination of claim 53, wherein said at least one mTOR inhibitor is at least one rapalog mTOR inhibitor, at least one non-rapalog mTOR inhibitor or a combination thereof.

55. The combination of claim 53, wherein said at least one mTOR inhibitor is represented by at least one of Formulas I, II, III, IV, V, VI, VII, VIII, IX, X, XI, XII, XIII, XIV, XV, XVI, XVII, a solvate, a hydrate, a stereoisomer, a pharmaceutically acceptable prodrug, a pharmaceutically active metabolite, a pharmaceutically acceptable salt, a crystalline form, an amorphous form, a physiologically functional derivative or a combination thereof optionally, wherein said at least one mTOR inhibitor is represented by at least one of Formulas I, II, III, a solvate, a hydrate, a stereoisomer, a pharmaceutically acceptable prodrug, a pharmaceutically active metabolite, a pharmaceutically acceptable salt, a crystalline form, an amorphous form, a physiologically functional derivative or a combination thereof.

56. The combination of claim 52, wherein said agonist of AMHR2 is at leas one of AMH protein, a derivative, an analogue, a variant or a homologue of said AMH protein, optionally wherein said AMH is human AMH (hAMH).

57. The combination of claim 56, wherein said derivative of an AMH protein is or comprises a fragment or peptide thereof an analogue, a variant or a homologue of the fragment or peptide thereof.

58. The combination of claim 56, wherein said hAMH is or comprises a C-terminus region of hAMH or any derivative, analogue or fragment thereof.

59. The combination of claim 56, wherein said hAMH is or comprises an amino acid sequence denoted by at least one of SEQ ID NO:1, SEQ ID NO:3, SEQ ID NO:4, SEQ ID NO:5 or SEQ ID NO:6.

60. A method for retaining Primordial Follicle (PMF) reserve in a mammalian subject, the method comprising administering to the subject a therapeutically effective amount of a combination comprising at least two modulators, wherein at least one of the at least two modulators is an mTOR inhibitor and at least one other of the at least two modulators is an agonist of AMHR2.

61. The method of claim 60, wherein said method results in at least one of inhibition of primordial follicles activation, inhibition of ovarian follicles depletion, inhibition of premature ovarian insufficiency (POI), destruction of the oocyte pool, reducing follicle apoptosis or any combination thereof.

62. The method of claim 60, for treating, inhibiting, preventing or ameliorating infertility or infertility-related conditions in the mammalian subject.

63. The method of claim 60, wherein said at least one mTOR inhibitor is a rapalog mTOR inhibitor, a non-rapalog mTOR inhibitor or a combination thereof.

64. The method claim 60, wherein said at least one mTOR inhibitor is represented by at least one of Formulas I, II, III, IV, V, VI, VII, VIII, IX, X, XI, XII, XIII, XIV, XV, XVI, XVII, a solvate, a hydrate, a stereoisomer, a pharmaceutically acceptable prodrug, a pharmaceutically active metabolite, a pharmaceutically acceptable salt, a crystalline form, an amorphous form, a physiologically functional derivative or a combination thereof, optionally said at least one mTOR inhibitor is represented by at least one of Formulas I, II, III a solvate, a hydrate, a stereoisomer, a pharmaceutically acceptable prodrug, a pharmaceutically active metabolite, a pharmaceutically acceptable salt, a crystalline form, an amorphous form, a physiologically functional derivative or a combination thereof.

65. The method of claim 60, wherein said agonist of AMHR2 is at leas one of AMH protein, a derivative, an analogue, a variant or a homologue of the protein optionally wherein said AMH is human AMH (hAMH).

66. The method of claim 65, wherein said hAMH is or comprises a C-terminus region of hAMH or any derivative, analogue or fragment thereof.

67. The method of claim 65, wherein said hAMH is or comprises a polypeptide denoted by at least one of SEQ ID NO:1, SEQ ID NO:3, SEQ ID NO:4, SEQ ID NO:5 or SEQ ID NO:6.

68. The method of claim 60, wherein said subject is suffering from at least one of (i) at least one of primordial follicles activation, ovarian follicles depletion, POI, destruction of the oocyte pool, follicle apoptosis, reduced fertility or any combinations thereof and/or (ii) at least one of an autoimmune disorder, a metabolic disorder, an infection, an environmental factor, a medical procedure, or any combinations thereof.

69. The method of claim 68, wherein said medical procedure is an anti-cancer treatment, optionally said anti-cancer treatment is at least one of chemotherapy, radiotherapy or a combination thereof.

70. The method of claim 60, wherein said subject is or was diagnosed with cancer and was treated or is treated with chemotherapy.

71. The method of claim 60, the method comprising (i) administering at least one mTOR inhibitor and said agonist of AMHR2 simultaneously, (ii) administering said at least one mTOR inhibitor prior to administration of the agonist of AMHR2 or (iii) administering said at least one mTOR inhibitor after administration of the agonist of AMHR2.


SEQUENCE LISTING (6 sequences)

SEQ ID NO: 1 - protein, 560 aa, Homo sapiens
MRDLPLTSLALVLSALGALLGTEALRAEEPAVGTSGLIFREDLDWPPGSPQEPLCLVALGGDSNGSSSPLRVVGALSAYEQAFLGAVQRARWGPRDLATFGVCNTGDRQAALPSLRRLGAWLRDPGGQRLVVLHLEEVTWEPTPSLRFQEPPPGGAGPPELALLVLYPGPGPEVTVTRAGLPGAQSLCPSRDTRYLVLAVDRPAGAWRGSGLALTLQPRGEDSRLSTARLQALLFGDDHRCFTRMTPALLLLPRSEPAPLPAHGQLDTVPFPPPRPSAELEESPPSADPFLETLTRLVRALRVPPARASAPRLALDPDALAGFPQGLVNLSDPAALERLLDGEEPLLLLLRPTAATTGDPAPLHDPTSAPWATALARRVAAELQAAAAELRSLPGLPPATAPLLARLLALCPGGPGGLGDPLRALLLLKALQGLRVEWRGRDPRGPGRAQRSAGATAADGPCALRELSVDLRAERSVLIPETYQANNCQGVCGWPQSDRNPRYGNHVVLLLKMQVRGAALARPPCCVPTAYAGKLLISLSEERISAHHVPNMVATECGCR

SEQ ID NO: 2 - DNA, 1809 bp, Homo sapiens
  source 1..1809: mol_type = other DNA
agcacccacgatgcgggacctgcctctcaccagcctggccctagtgctgtctgccctgggggctctgctggggactgaggccctcagagcagaggagccagctgtgggcaccagtggcctcatcttccgagaagacttggactggcctccaggcagcccacaagagcctctgtgcctggtggcactgggcggggacagcaatggcagcagctcccccctgcgggtggtgggggctctaagcgcctatgagcaggccttcctgggggccgtgcagagggcccgctggggcccccgagacctggccaccttcggggtctgcaacaccggtgacaggcaggctgccttgccctctctacggcggctgggggcctggctgcgggaccctggggggcagcgcctggtggtcctacacctggaggaagtgacctgggagccaacaccctcgctgaggttccaggagcccccgcctggaggagctggccccccagagctggcgctgctggtgctgtaccctgggcctggccctgaggtcactgtgacgagggctgggctgccgggtgcccagagcctctgcccctcccgagacacccgctacctggtgttagcggtggaccgccctgcgggggcctggcgcggctccgggctggccttgaccctgcagccccgcggagaggactcccggctgagtaccgcccggctgcaggcactgctgttcggcgacgaccaccgctgcttcacacggatgaccccggccctgctcctgctgccgcggtccgagcccgcgccgctgcctgcgcacggccagctggacaccgtgcccttcccgccgcccaggccatccgcggaactcgaggagtcgccacccagcgcagaccccttcctggagacgctcacgcgcctggtgcgggcgctgcgggtccccccggcccgggcctccgcgccgcgcctggccctggatccggacgcgctggccggcttcccgcagggcctagtcaacctgtcggaccccgcggcgctggagcgcctactcgacggcgaggagccgctgctgctgctgctgaggcccactgcggccaccaccggggatcctgcgcccctgcacgaccccacgtcggcgccgtgggccacggccctggcgcgccgcgtggctgctgaactgcaagcggcggctgccgagctgcgaagcctcccgggtctgcctccggccacagccccgctgctggcgcgcctgctcgcgctctgcccaggtggccccggcggcctcggcgatcccctgcgagcgctgctgctcctgaaggcgctgcagggcctgcgcgtggagtggcgcgggcgggatccgcgcgggccgggtcgggcacagcgcagcgcgggggccaccgccgccgacgggccgtgcgcgctgcgcgagctcagcgtagacctccgcgccgagcgctccgtactcatccccgagacctaccaggccaacaattgccagggcgtgtgcggctggcctcagtccgaccgcaacccgcgctacggcaaccacgtggtgctgctgctgaagatgcaggtccgtggggccgccctggcgcgcccaccctgctgcgtgcccaccgcctacgcgggcaagctgctcatcagcctgtcggaggagcgcatcagcgcgcaccacgtgcccaacatggtggccaccgagtgtggctgccggtgacccctgcgccgcgcggactcctgccccgagggtccggacgcgccccagctcgcgccccttcccatatttattcggaccccaagcatcgccccaataaagaccagcaagcaaccggc

SEQ ID NO: 3 - protein, 99 aa, synthetic construct
CALRELSVDLRAERSVLIPETYQANNCQGVCGWPQSDRNPRYGNHVVLLLKMQVRGAALARPPCCVPTAYAGKLLISLSEERISAHHVPNMVATECGCR

SEQ ID NO: 4 - protein, 109 aa, synthetic construct
SAGATAADGPCALRELSVDLRAERSVLIPETYQANNCQGVCGWPQSDRNPRYGNHVVLLLKMQVRGAALARPPCCVPTAYAGKLLISLSEERISAHHVPNMVATECGCR

SEQ ID NO: 5 - protein, 110 aa, synthetic construct
RSAGATAADGPCALRELSVDLRAERSVLIPETYQANNCQGVCGWPQSDRNPRYGNHVVLLLKMQVRGAALARPPCCVPTAYAGKLLISLSEERISAHHVPNMVATECGCR

SEQ ID NO: 6 - protein, 108 aa, synthetic construct
AGATAADGPCALRELSVDLRAERSVLIPETYQANNCQGVCGWPQSDRNPRYGNHVVLLLKMQVRGAALARPPCCVPTAYAGKLLISLSEERISAHHVPNMVATECGCR